SEQUENCE LISTING (47 sequences)

SEQ ID NO: 1 - protein, 19 aa, Artificial Sequence
  Pilot peptide
MFPTIPLSRLFDNAMLRAH

SEQ ID NO: 2 - protein, 25 aa, Artificial Sequence
  Q11; synthesized
AGTKPQGKPASNLVECVFSLFKKCN

SEQ ID NO: 3 - protein, 19 aa, Artificial Sequence
  Q28; synthesized
DIRHRINNSIWRDIFLKRK

SEQ ID NO: 4 - protein, 15 aa, Artificial Sequence
  Q30; synthesized
DLRGVPNPWGWIFGR

SEQ ID NO: 5 - protein, 14 aa, Artificial Sequence
  Q31; synthesized
DLRNIFLKIKFKKK

SEQ ID NO: 6 - protein, 17 aa, Artificial Sequence
  Q45; synthesized
EMRISRIILDFLFLRKK

SEQ ID NO: 7 - protein, 16 aa, Artificial Sequence
  Q46; synthesized
EMRKSNNNFFHFLRRI

SEQ ID NO: 8 - protein, 17 aa, Artificial Sequence
  Q47; synthesized
EMRLPKILRDFIFPRKK

SEQ ID NO: 9 - protein, 17 aa, Artificial Sequence
  Q53; synthesized
ESRLPKILLDFLFLRKK

SEQ ID NO: 10 - protein, 17 aa, Artificial Sequence
  Q54; synthesized
ESRLPKIRFDFIFPRKK

SEQ ID NO: 11 - protein, 16 aa, Artificial Sequence
  Q58; synthesized
DSRIRMGFDFSKLFGK

SEQ ID NO: 12 - protein, 27 aa, Artificial Sequence
  Q101; synthesized
GLWEDILYSLNIIKHNNTKGLHHPIQL

SEQ ID NO: 13 - protein, 18 aa, Artificial Sequence
  synthesized
GLWEDLLYNINRYAHYIT

SEQ ID NO: 14 - protein, 18 aa, Artificial Sequence
  Q171; synthesized
SGSLSTFFRLFNRSFTQA

SEQ ID NO: 15 - protein, 21 aa, Artificial Sequence
  synthesized
SGSLSTFFRLFNRSFTQALGK

SEQ ID NO: 16 - protein, 5 aa, Artificial Sequence
  Q19; synthesized
NNWNN

SEQ ID NO: 17 - protein, 3 aa, Artificial Sequence
  Q155; synthesized
NWN

SEQ ID NO: 18 - protein, 7 aa, Artificial Sequence
  Q13; synthesized
AIFILAS

SEQ ID NO: 19 - protein, 7 aa, Artificial Sequence
  Q14; synthesized
AITLIFI

SEQ ID NO: 20 - protein, 8 aa, Artificial Sequence
  Q17; synthesized
ALILTLVS

SEQ ID NO: 21 - protein, 8 aa, Artificial Sequence
  Q132; synthesized
LFSLVLAG

SEQ ID NO: 22 - protein, 8 aa, Artificial Sequence
  Q133; synthesized
LFVVTLVG

SEQ ID NO: 23 - protein, 7 aa, Artificial Sequence
  Q137; synthesized
LVTLVFV

SEQ ID NO: 24 - protein, 7 aa, Artificial Sequence
  Q184; synthesized
SIFTLVA

SEQ ID NO: 25 - protein, 8 aa, Artificial Sequence
  Q210; synthesized
VAVLVLGA

SEQ ID NO: 26 - protein, 28 aa, Artificial Sequence
  Q49; synthesized
EQLSFTSIGILQLLTIGTRSCWFFYCRY

SEQ ID NO: 27 - protein, 26 aa, Artificial Sequence
  Q125; synthesized
KSSAYSLQMGATAIKQVKKLFKKWGW

SEQ ID NO: 28 - protein, 19 aa, Artificial Sequence
  Q138; synthesized
MAGNSSNFIHKIKQIFTHR

SEQ ID NO: 29 - protein, 23 aa, Artificial Sequence
  Q208; synthesized
TNRNYGKPNKDIGTCIWSGFRHC

SEQ ID NO: 30 - protein, 7 aa, Artificial Sequence
  Q206; synthesized
SYPGWSW

SEQ ID NO: 31 - protein, 5 aa, Artificial Sequence
  Q34; synthesized
DRVGA

SEQ ID NO: 32 - protein, 5 aa, Artificial Sequence
  Q50; synthesized
ERGMT

SEQ ID NO: 33 - protein, 5 aa, Artificial Sequence
  Q52; synthesized
ERPVG

SEQ ID NO: 34 - protein, 5 aa, Artificial Sequence
  Q160; synthesized
QKGMY

SEQ ID NO: 35 - protein, 5 aa, Artificial Sequence
  Q162; synthesized
QRGMI

SEQ ID NO: 36 - protein, 5 aa, Artificial Sequence
  Q192; synthesized
SRNAT

SEQ ID NO: 37 - protein, 5 aa, Artificial Sequence
  Q192; synthesized
SRNVT

SEQ ID NO: 38 - protein, 12 aa, Artificial Sequence
  Constituent peptide isolated and sequenced from diluent
THEMETHVADLS

SEQ ID NO: 39 - protein, 12 aa, Artificial Sequence
  Constituent peptide isolated and sequenced from diluent
HTEMETHVADLS

SEQ ID NO: 40 - protein, 12 aa, Artificial Sequence
  Constituent peptide isolated and sequenced from diluent
THEMETHAVDLS

SEQ ID NO: 41 - protein, 13 aa, Artificial Sequence
  Constituent peptide isolated and sequenced from diluent
WVNDNEEGFFSLN

SEQ ID NO: 42 - protein, 13 aa, Artificial Sequence
  Constituent peptide isolated and sequenced from diluent
WVNDNEEGFFSAR

SEQ ID NO: 43 - protein, 14 aa, Artificial Sequence
  Constituent peptide isolated and sequenced from diluent
ADVNDNEEGFFSAR

SEQ ID NO: 44 - protein, 13 aa, Artificial Sequence
  Constituent peptide isolated and sequenced from diluent
ERNDNEEGFFSAR

SEQ ID NO: 45 - protein, 14 aa, Artificial Sequence
  Constituent peptide isolated and sequenced from diluent
AVDNDNEEGFFSAR

SEQ ID NO: 46 - protein, 13 aa, Artificial Sequence
  Constituent peptide isolated and sequenced from diluent
QRDDNEEGFFSAR

SEQ ID NO: 47 - protein, 14 aa, Artificial Sequence
  Constituent peptide isolated and sequenced from diluent
QAEADNEEGFFSAR

(19) United States
(12) Patent Application Publication
(10) Pub. No.: US 2023/0089604 A1
(43) Pub. Date: Mar. 23, 2023

(54) ANTI-ADSORPTION SOLUTION

(71) Applicant: Universiteit Gent, Gent (BE)

(72) Inventors: Bart De Spiegeleer, Gent (BE); Frederick Verbeke, Gent (BE); Nathalie Bracke, Zele (BE); Evelien Wynendaele, Herzele (BE)

(73) Assignee: Universiteit Gent, Gent (BE)

(21) Appl. No.: 17/763,003

(22) PCT Filed: Sep. 24, 2020
(86) PCT No.: PCT/EP2020/076754

§ 371 Date: Mar. 23, 2022

(30) Foreign Application Priority Data
Sep. 24, 2019 (EP) 19199367.4

(51) Int. Cl. G01N 33/68

(52) U.S. Cl. CPC G01N 33/6848

(57) ABSTRACT

The present invention is directed to an anti-adsorption solution for the analysis of peptides. Also a process for the preparation of such an anti-adsorption solution and the use of said anti-adsorption solution are described.

### FIELD OF THE INVENTION

The present invention is directed to an anti-adsorption solution for the analysis of peptides. Also a process for the preparation of such an anti-adsorption solution and the use of said anti-adsorption solution are described.

### BACKGROUND TO THE INVENTION

Peptides, hovering in between the small molecules and proteins, are considered a promising class of compounds in the biomedical field. Here, targeted as well as untargeted peptidomics is diverted towards diagnostic applications as well as (possible) therapeutics due to their inherent high target affinity, unrevoked selectivity and generally low toxicity [1]. Accordingly, there is the necessity to develop suitable peptide (bio)-analytical methods. The physicochemical properties that make peptides such a promising class comes with the side-effect of often a challenging analytical method development. A key hurdle in this method development is the unpredictable adsorption of the peptide(s) of interest to plastic and glass consumables occurring from the pre-analytical to the analytical phase. Especially at low concentrations, this adsorption is more pronounced, often exhibiting a non-linear nature and finally resulting in unwanted elevated limits of detection and quantification as well as unreproducible results. Hence, the biologically advantageous heterogeneity in peptide (physico)chemical properties can become a cumbersome characteristic for analytical purposes. Additionally, this heterogeneity inherently comes with the consequence that one-fits-all approaches are frequently impossible [2-5]. Various approaches to overcome this peptide adsorption have already been reported, including organic solvents addition, pH alteration, specific vial type (low adsorption glass and plastic consumables due to surface modifications), use of surfactants and adsorption competitors [5].

Adsorption competition entails occupation of those physicochemical moieties on the consumables that would otherwise be available to the peptide analyte and hence withdraw it from the analytical solution. Based on the specific interactions, various approaches are already reported; for example, a structural analogue of the considered peptide, hence occupying the peptide-specific adsorption locations [6]. Since customized peptide synthesis comes with a price, this dedicated approach is cumbersome and expensive. Other adsorption competitors, also referred to as carrier proteins or displacement agents [5], are the addition of 0.05% V/V rat plasma [7], bovine serum albumin (e.g. 1% m/V) [8, 9], Fmoc-arginine [10], poly(ethylenimine) [11], glucagon [12, 13], diluted SPE-purified bovine plasma [14] or structurally analogue isotope-labeled peptides/proteins [15]. Physicochemical modifications of the consumable surface via siliconizing agents [9, 16] or polyethylene glycol [17] have also been reported. However, Goebel-Stengel et al. demonstrated decreased recoveries of peptides when the glass containers were siliconized [9]. Bovine serum albumin addition has proven a valuable approach to overcome adsorption, however hindering direct LC-MS analysis and increasing the likelihood of adsorption or inclusion of the peptide to albumin, a protein notoriously known for its binding properties.

Another anti-adsorption approach is coating of the surface of the vials or flasks with silicon-based agents, or polyethylene glycol, although said coating methods have not always been shown to be successful.

Together, the currently available methods for reducing peptide adsorption to their surface all have their disadvantages. For example, the addition of bovine serum albumin can be an easy solution to reduce the peptide adsorption, but on the other hand, the presence of bovine serum albumin also complicates analysis afterwards, such as mass spectroscopy, of the peptide solution and also increases the potential adsorption, binding or inclusion of the peptides to the albumin.

### SUMMARY OF THE INVENTION

In the present invention, a novel anti-adsorption solution for the analysis of peptides and a process for the preparation of such a solution was identified. Typical for the invention, is that despite the presence of anti-adsorption proteins and/or peptides, such as for example bovine serum albumin, the further analysis of the peptides of interest is not influenced. The anti-adsorption solution of the present invention is typically characterized in that the anti-adsorption proteins and/or peptides in the solution are precipitated or eliminated and further diluted. Optionally a heating step before precipitation can take place.

Peptide analysis in the low concentration range can be challenging due to the adsorption of the peptides of interest to the analytical vials. Various anti-adsorption approaches are already reported, though with limited success. In the present application, the inventors found that by creating a mixture of the anti-adsorption peptides, such as obtained from bovine serum albumin, the anti-adsorption solution becomes compatible for use in peptide analytical methods, and no interference or only very limited interference with the peptides of interest (e.g. present in a sample) occurs. In the process for preparing an anti-adsorption solution of the present invention, a controlled mixture of peptides is combined with precipitation of the anti-adsorption proteins and/or peptides. As a result a novel anti-adsorption solution is obtained that remarkably decreases the adsorption of the peptides of interest to the plastic or glass vial. Additionally, the anti-adsorption solution according to the present invention is easily prepared, is compatible with organic solvent addition and/or pH alteration to increase its use-flexibility.

### BRIEF DESCRIPTION OF THE DRAWINGS

FIG. 1: Peptide stability of peptide MFPTIPLSRLFDNAMLRAH (1 mg/mL). A) blank: 50/50 V/V water/acetonitrile+0.1% m/V formic acid; B) MFPTIPLSRLFDNAMLRAH at a concentration of 1 mg/mL in water/acetonitrile 50/50 V/V+0.1% m/V formic acid at 0 h, and C) MFPTIPLSRLFDNAMLRAH at a concentration of 1 mg/mL in water/acetonitrile 50/50 V/V+0.1% m/V formic acid at 3 h.

FIG. 2. Glass adsorption of peptide MFPTIPLSRLFDNAMLRAH with peptide peak area in function of incubation time. Peptide stored in glass vial insert (100 ng/mL; diluent 50/50 (V/V) water/acetonitrile+0.1% m/V formic acid).

FIG. 3. Effect of anti-adsorption diluent on peptide recovery (amino acid sequence: MFPTIPLSRLFDNAMLRAH) peak area in function of time, when stored in a glass vial insert at a concentration of 100 ng/mL. Solvent 1: 50/50 (V/V) acetonitrile/water+0.1% m/V formic acid; solvent 2: anti-adsorption diluent Source 1. Recovery expressed as percentage of the peptide peak area at 0 min in solvent 1.

FIG. 4. Hierarchal cluster analysis (right) of 36 model peptides with accompanying heat map (left) of the predicted, most suitable condition to minimize glass adsorption. Peptide ID according to Wynendaele et al. [18]. Color code from light to dark grey (see legend); with 0 representing 0% V/V of the concerned solvent and 1 representing 100% V/V of the concerned solvent. The color codes of the peptides concur with the 3 “physicochemical” clusters observed via Principal Component Analysis (see FIG. 5). Anti-adsorption diluent Source 1 was used during this experiment.

FIG. 5. Effect of anti-adsorption diluent Source 1 on peptide glass vial adsorption of a peptide (amino acid sequence: EQLSFTSIGILQLLTIGTRSCWFFYCRY). (A) 1 nM without anti-adsorption diluent giving a peptide peak area of 1363.8; (B) 1 nM with anti-adsorption diluent Source 1 giving a peptide peak area of 16772.4.

FIG. 6. Score plot of model peptides showing 3 clusters (A-C) obtained by Principal Component Analysis (PCA) PC1 versus PC2 (upper panel). PCA of PC1 versus PC3 (lower panel). Grey ellipse represents Hotelling's T2 95% confidence interval.

FIG. 7: Comparison of selected descriptors amongst cluster D and E peptides in proof of concept experiment. A) Molecular weight; B) Isoelectric point; C) Log P; D) Hydrogen bound donors and E) Hydrogen bound acceptors.

FIG. 8: Representative images (n=3) of matrix effects caused by AAD originating from 3 different sources on the peptide EMRKSNNNFFHFLRRI. A) blank, i.e. solvent (50/50 V/V water/acetonitrile+0.1% m/V formic acid); B) anti-adsorption diluent Source 1; C) anti-adsorption diluent Source 2; and D) anti-adsorption diluent Source 3. An arrow indicates the retention time of the peptide if chromatographed.

FIG. 9. Glass adsorption of peptide MFPTIPLSRLFDNAMLRAH (100 ng/mL) examined via consecutive transfer of the analytical solution to inserts with solvent being 50/50 V/V water/acetonitrile+0.1% m/V formic acid or anti-adsorption diluent (AAD). Panel A: no transfer to new insert and panel B: consecutive transfer to a total of 5 inserts with analysis of insert number 5. Error bars represent standard deviation (n=3).

FIG. 10: Representative chromatograms of peptide MFPTIPLSRLFDNAMLRAH (100 ng/mL) glass adsorption study (see FIG. 6). Panels A and C no transfer and panels B and D repetitive transfer to a total of 5 inserts with analysis of insert number 5. Panels A and B: solvent (50/50 V/V water/acetonitrile+0.1% m/V formic acid; Panels C and D: anti-adsorption diluent batch 1.

FIG. 11. Adsorption of peptide MFPTIPLSRLFDNAMLRAH (100 ng/mL) to glass and polypropylene inserts in the presence of absence of anti-adsorption diluent (AAD) Source 1. Experiment performed in triplicate with standard deviation as error bars.

FIG. 12. Boxplots demonstrating the impact of organic solvent and/or protein source on the functionality of the anti-adsorption solution for Q46 (5 nM). BSA stands for bovine serum albumin, LAC stands for lactalbumin, OVAL stands for ovalbumin, ACN stands for acetonitrile, EtOH stands for denatured ethanol and IPA stands for isopropanol. All organic solvents and water contained 0.1% m/V formic acid. Data are depicted as boxplots of 6 technical replicates (except ACN BSA (n=5 due to 1 outlier)) relative to BSA with FA (average is 100% control).

FIG. 13. Impact of 0.1% m/V trifluoroacetic acid on the functionality of the anti-adsorption solution for Q46 (5 nM). BSA stands for bovine serum albumin, FA for formic acid, TFA stands for trifluoroacetic acid. Data are depicted as dotted boxplots (6 technical replicates) relative to BSA with FA (average is 100% control).

FIG. 14. Impact of acetonitrile percentage in step 2 of the production process on the functionality of the anti-adsorption solution for Q46 (5 nM). BSA with FA equals to the previously reported AAD [1], containing 75% V/V acetonitrile supplemented with 0.1% m/V formic acid. BSA stands for bovine serum albumin, FA for formic acid, 50 and 90 indicate the respective % V/V of acetonitrile supplemented with 0.1% m/V formic acid in step 2 of the production process, the remaining is made up with water+0.1% m/V formic acid. Data are depicted as boxplots of 6 technical replicates relative to BSA with FA (average is 100% control).

FIG. 15. Impact of formic acid on the functionality of the anti-adsorption solution for Q46 (5 nM). BSA stand for bovine serum albumin and FA for formic acid. Data are depicted as box-plots (6 technical replicates) with the BSA with FA as the control (average=100%).

FIG. 16. Impact of heating on AAD functionality for Q46 (5 nM). BSA stands for bovine serum albumin, and FA for formic acid. Data are depicted as boxplots of 6 technical replicates relative to BSA with FA (average is 100% control).

### DETAILED DESCRIPTION OF THE INVENTION

In the present invention, a novel solution for the analysis of peptides and a process for the preparation of such a solution was identified. Typical for the invention, is that despite the presence of anti-adsorption proteins and/or peptides, the further analysis of the peptides of interest is not influenced. The anti-adsorption solution of the present invention is typically characterized in that the anti-adsorption proteins and peptides in the solution are precipitated or eliminated and further diluted. Optionally a heating step can be performed before precipitation or elimination of the protein.

In a first aspect of the invention a process for the preparation of an anti-adsorption solution or diluent is provided. The anti-adsorption diluent comprises a mixture of anti-adsorption components, including peptides, and is obtained by precipitating the protein with acetonitrile. Optionally the protein solution can be boiled before precipitation of the protein. In one embodiment, the protein is albumin, ovalbumin or lactalbumin, in particular bovine serum albumin (BSA).

In general the process of the invention comprises the following steps: 1) preparation of a solution of one or more proteins (or optionally peptides) (also referred herein as one or more anti-adsorption proteins or optionally anti-adsorption peptides) optionally supplemented with a first acid, 2) dilution of the solution of step 1 by the addition of an organic solvent solution optionally comprising a second acid; 3) optionally heating the solution obtained in step 2; 4) cooling down the solution obtained in step 2 or 3; 5) separation and isolation of the solution obtained in step 4 from any precipitate formed during steps 1 to 4; and 6) dilution of the solution obtained in step 5 in water or any other suitable solvent optionally supplemented with a third acid, thereby obtaining the anti-adsorption solution.

In a specific embodiment, the process of the invention comprises the following steps: 1) preparation of a solution of one or more proteins (or optionally peptides) (also referred herein as one or more anti-adsorption proteins or optionally anti-adsorption peptides), 2) dilution of the solution of step 1 by the addition of an organic solvent solution; 3) optionally heating the solution obtained in step 2; 4) cooling down the solution obtained in step 2 or 3; 5) separation and isolation of the solution obtained in step 4 from any precipitate formed during steps 1 to 4; and 6) dilution of the solution obtained in step 5 in water or any other suitable solvent, thereby obtaining the anti-adsorption solution.

In another specific embodiment, the process of the invention comprises the following steps: 1) preparation of a solution of one or more proteins (or optionally peptides) (also referred herein as one or more anti-adsorption proteins or optionally anti-adsorption peptides) supplemented with a first acid, 2) dilution of the solution of step 1 by the addition of an organic solvent solution comprising a second acid; 3) optionally heating the solution obtained in step 2; 4) cooling down the solution obtained in step 2 or 3; 5) separation and isolation of the solution obtained in step 4 from any precipitate formed during steps 1 to 4; and 6) dilution of the solution obtained in step 5 in water or any other suitable solvent supplemented with a third acid, thereby obtaining the anti-adsorption solution.

By using the process of the present invention, the protein in the solution is (optionally) heated, precipitated or eliminated and the resulting remaining solution finally diluted. As a result, the protein is expected to be partially and selectively hydrolyzed, while the bulk protein is precipitated, and the resulting constituents, consisting of i.a. peptides, maintain their anti-adsorptive capacity, but their analytical interference with the peptides of interest is reduced or even completely absent.

In the process of the present invention, a solution of 25 ng/ml to 1 g/ml of one or more anti-adsorption proteins and/or peptides in water is prepared in step 1 and optionally supplemented with 0.1% to 10% m/V of a first acid. In a further embodiment, a solution of 25 μg/ml to 25 mg/ml of one or more anti-adsorption proteins and/or peptides in water is prepared in step 1 and optionally further supplemented with 0.1% to 10%; preferably 0.1% to 1% of the first acid. For example, a solution of 10 mg/ml of one or more anti-adsorption proteins and/or peptides in water is prepared and further supplemented with 0.1% of the first acid. In another example, a solution of 10 mg/ml of one or more anti-adsorption proteins and/or peptides in water is prepared without the addition of an acid.

In a further aspect, and in step 2 of the process of the present invention, the solution prepared in step 1 is diluted by the addition of a 0% to 99% V/V organic solvent solution in order to obtain a concentration of 2.5 ng/ml to 0.1 g/ml of one or more proteins and/or peptides in the solution. Optionally, the organic solvent may comprise 0.1% to 10% (m/V) of a second acid. In still a further aspect, in step 2, the solution prepared in step 1 is diluted to a concentration of 2.5 ng/ml to 0.1 g/ml of one or more anti-adsorption proteins and/or peptides by the addition of a 0% to 99% V/V organic solvent solution optionally supplemented with 0.1% to 10% (m/V) of the second acid. In an even more preferred embodiment, the solution prepared in step 1 is diluted to a concentration of 2.5 ng/ml of one or more anti-adsorption proteins and/or peptides by the addition of a 95% (V/V) organic solvent solution with 0.1% (m/V) of the second acid.

In the optional step 3 of the process according to the present invention, the solution obtained in step 2 is heated. In further embodiment, the solution is heated for 1 to 1000 minutes to 30° C. to 120° C. In a preferred embodiment, in step 3, the solution obtained in step 2 is heated for 1 to 10, in particular 5 to 10, minutes to 75° C. to 100° C. In an even more preferred embodiment, the solution is heated for 5 minutes to 95° C. In a particular and preferred embodiment, the method of the present invention comprises step 3 according to all its different embodiments, and hence step 3 is not optional, but is always included in the method of the present invention. In still another further embodiment, step 3 is optional in the method according to the present invention, and the method according to the invention can be performed without step 3.

In the process according to the present invention, the solution obtained in step 2 or 3 is cooled down in step 4. In a further aspect, in step 4 the solution is cooled down for 0 to 600 minutes to 40° C. to −35° C. In a preferred embodiment, in step 4, the solution obtained in step 3 is cooled down for 5 to 60 minutes to 8° C. to −8° C. In an even more preferred embodiment, the solution is cooled down at 0° C. on ice for 30 minutes.

The process of the present invention is further characterized in that it comprises a step 5 wherein from the solution of step 4 any precipitate formed during steps 1 to 4 is separated and isolated. Said separation and isolation of the solution from any precipitate can be performed by any suitable separation method, such as for example centrifugation or filtration. In a preferred embodiment, the separation and isolation is performed by centrifugation. In an even more preferred embodiment, the centrifugation in step 5 is performed for 0 to 600 minutes at 100 g to 100000 g. In an even more preferred embodiment, the centrifugation in step 5 is performed for 1 to 60 minutes, for example for 10, 20 or 30 minutes, at 1000 g to 50000 g. For example, the centrifugation in step 5 is performed for 15 minutes at 20000 g at 4° C.

In the last step (step 6) of the process according to the invention, the solution obtained from step 5 is diluted in water optionally supplemented with a third acid, thereby obtaining the anti-adsorption solution. In a preferred embodiment, in step 6, the solution is diluted in water supplemented with 0.1% to 10% (m/V) of a third acid; preferably diluted in water supplemented with 0.1% to 1% (m/V) of a third acid, such as for example 0.1%, 0.2% or 0.5% of a third acid. In a most preferred embodiment, 0.1% of a third acid is used. In further embodiment, in step 6, the solution obtained in step 5 is diluted in water supplemented with said third acid by the addition of 0.1 to 9.9 parts of the solution obtained in step 5 in 9.9 to 0.1 parts water supplemented with said third acid. In a preferred embodiment, in step 6, the solution obtained in step 5 is diluted in water supplemented with the third acid by the addition of 2 parts of the solution on 1 part of water supplemented with said third acid. In another embodiment, in step 6, the solution is diluted in water without the presence of any acid. In particular, the solution obtained in step 5 is diluted in water by the addition of 0.1 to 9.9 parts of the solution obtained in step 5 in 9.9 to 0.1 parts water. In a preferred embodiment, in step 6, the solution obtained in step 5 is diluted in water by the addition of 2 parts of the solution in 1 part of water.

As already mentioned above, the present invention provides a process for the preparation of an anti-adsorption solution starting from a solution of one or more proteins, including peptides, in water (or other suitable excipient or solvent) optionally supplemented with a first acid. Said one or more proteins and/or peptides can be selected from albumin, ovalbumin, lactalbumin globulin, gelatin, or any other protein or peptide source, or a combination thereof. In a preferred embodiment, the process of the present invention starts from a solution of one or more anti-adsorption proteins, including peptides, in water (or other suitable solvent) optionally supplemented with a first acid, wherein said solution comprises at least albumin, preferably at serum albumin; even more preferably at least bovine serum albumin, as anti-adsorption protein. In an even more preferred embodiment, the process of the present invention starts from a solution of albumin, preferably serum albumin, even more preferably bovine serum albumin, in a solvent such as water supplemented with a first acid.

Thus, in a particular aspect, the present invention provides a process for the preparation of an anti-adsorption solution, said process comprising the following steps: 1) preparation of a solution of 25 μg/ml to 25 mg/ml serum albumin in water optionally supplemented with 0.1% to 1% (m/V) of a first acid, 2) dilution of the solution of step 1 to a concentration of 2.5 μg/ml to 2.5 mg/ml serum albumin by the addition of 0% to 95% (V/V) of an organic solvent solution optionally comprising 0.1% to 1% of a second acid; 3) optionally heating the solution obtained in step 2 for 1 to 10 minutes to 75° C. to 100° C., 4) cooling down the solution of step 2 or 3 for 5 to 60 minutes to 8° C. to −8° C.; 5) separation and isolation of the solution obtained in step 4 from any precipitate formed during steps 1 to 4 by centrifugation; and 6) dilution of the solution obtained in step 5 in water optionally supplemented with 0.1% to 1% m/V of a third acid, thereby obtaining the anti-adsorption solution. In a further aspect, and in step 6, the solution obtained in step 5 is diluted in water optionally supplemented with the third acid by the addition of 0.1 to 9.9 parts of the solution obtained in step 4 or 5 in 9.9 to 0.1 parts water supplemented with said third acid. For example, in step 6, the solution obtained in step 5 is diluted in water optionally supplemented with the third acid by the addition of 2 parts of the solution on 1 part of water optionally supplemented with said third acid.

In another particular aspect, the present invention provides a process for the preparation of an anti-adsorption solution, said process comprising the following steps: 1) preparation of a solution of 25 μg/ml to 25 mg/ml serum albumin in water supplemented with 0.1% to 1% (m/V) of a first acid, 2) dilution of the solution of step 1 to a concentration of 2.5 μg/ml to 2.5 mg/ml serum albumin by the addition of 0% to 95% (V/V) of an organic solvent solution comprising 0.1% to 1% of a second acid; 3) optionally heating the solution obtained in step 2 for 1 to 10 minutes to 75° C. to 100° C., 4) cooling down the solution of step 2 or 3 for 5 to 60 minutes to 8° C. to −8° C.; 5) separation and isolation of the solution obtained in step 4 from any precipitate formed during steps 1 to 4 by centrifugation; and 6) dilution of the solution obtained in step 5 in water supplemented with 0.1% to 1% m/V of a third acid, thereby obtaining the anti-adsorption solution. In a further aspect, and in step 6, the solution obtained in step 5 is diluted in water supplemented with the third acid by the addition of 0.1 to 9.9 parts of the solution obtained in step 4 or 5 in 9.9 to 0.1 parts water with said third acid. For example, in step 6, the solution obtained in step 5 is diluted in water supplemented with the third acid by the addition of 2 parts of the solution on 1 part of water supplemented with said third acid.

In still another particular aspect, the present invention provides a process for the preparation of an anti-adsorption solution, said process comprising the following steps: 1) preparation of a solution of 25 μg/ml to 25 mg/ml serum albumin in water, 2) dilution of the solution of step 1 to a concentration of 2.5 μg/ml to 2.5 mg/ml serum albumin by the addition of 0% to 95% (V/V) of an organic solvent solution; 3) optionally heating the solution obtained in step 2 for 1 to 10 minutes to 75° C. to 100° C., 4) cooling down the solution of step 2 or 3 for 5 to 60 minutes to 8° C. to −8° C.; 5) separation and isolation of the solution obtained in step 4 from any precipitate formed during steps 1 to 4 by centrifugation; and 6) dilution of the solution obtained in step 5 in water, thereby obtaining the anti-adsorption solution. In a further aspect, and in step 6, the solution obtained in step 5 is diluted in water by the addition of 0.1 to 9.9 parts of the solution obtained in step 4 or 5 in 9.9 to 0.1 parts water. For example, in step 6, the solution obtained in step 5 is diluted in water by the addition of 2 parts of the solution on 1 part of water.

In the different embodiments of the process wherein an acid is used, the first acid, the second acid and the third acid are each independently selected from formic acid, acetic acid, trifluoro acetic acid or any other known acid. In yet a further embodiment, the first acid, the second acid and the third acid are the same. In an even more preferred embodiment, the first acid, the second acid and the third acid is formic acid.

Further, in all embodiments of the process of the present invention, an organic solvent is added to the solution in step 2. Said organic solvent is selected from acetonitrile, methanol, isopropanol, denatured ethanol or any known organic solvent. In a preferred embodiment, said organic solvent is acetonitrile, methanol, isopropanol, or denatured ethanol. In an even more preferred embodiment, said organic solvent is acetonitrile.

In a second aspect of the invention, an anti-adsorption solution is provided wherein said anti-anti-adsorption solution is obtained by the process according to any of the described embodiments.

In a third aspect, an anti-adsorption powder is disclosed wherein said powder is obtained by drying or freeze-drying of the anti-adsorption solution according to the invention.

In a further aspect, the combination of said anti-adsorption powder and a suitable solvent is provided. Said solvent can be selected from water or aqueous-solvent mixtures. In a particular embodiment, the combination of said anti-adsorption powder and a stabilized preservative (antioxidative inhibitors and/or antimicrobial inhibitors and/or enzyme inhibitors such as protease/peptidase inhibitors), such as for example sodium azide, is provided. In an even further embodiment, the anti-adsorption powder can be provided in a capsule or a tablet or any other form or formulation suitable for its practical intended use.

In a further aspect of the present invention, the use of an anti-adsorption solution or an anti-adsorption powder according to their different embodiments is provided for coating of a recipient such as a container, a vial, a tubing, a column, or any other analytical device. In a further embodiment, said recipient or column is made of glass or plastic. Still another further embodiment, said recipient or column is used in peptide analytical methods. Said peptide analytic methods are selected from immune-based protein analysis methods, such as ELISA, western blotting, liquid phase chromatography; mass spectrometry; or peptidomics. In a preferred embodiment, said peptide analytical methods are selected from liquid chromatography, mass spectrometry, and peptidomics.

In still another aspect of the invention, the use of an anti-adsorption solution or an anti-adsorption powder according to their different embodiments is provided for storage, detection, identification and/or separation of peptides.

A further aspect of the invention discloses the use of an anti-adsorption solution or an anti-adsorption powder according to their different embodiments in peptide analytical methods. Said peptide analytic methods are selected from immune-based protein analysis methods, such as ELISA, western blotting, liquid phase chromatography; mass spectrometry; or peptidomics. In a preferred embodiment, said peptide analytical methods are selected from liquid chromatography, mass spectrometry, and peptidomics. In a further embodiment, in said use, the anti-adsorption solution or powder is combined with a solvent; in particular an organic solvent.

As already said, the anti-adsorption solution or powder of the present invention is particularly useful in peptide analytic methods. Said peptides can be single peptides or mixtures of one or more peptides, and more specific, peptides present in a sample. Any sample can be analyzed, such as a bodily fluid sample derived from a subject. The sample can be blood, serum, nasal mucus, sputum, lung aspirate, vaginal fluid, gastric fluid, saliva, urine, faeces, cerebrospinal fluid. The subject is selected from a human or a non-human animal; preferably from a human, a non-human mammal, or a non-mammal. Said peptides can be hydrophobic peptides or hydrophilic peptides. In preferred embodiment, said peptides are hydrophilic peptides.

In a further aspect, the present invention provides a method for the separation and/or detection of peptides wherein the peptides are provided or diluted in an anti-adsorption solution according to the present invention followed by a peptide analytical method; preferably followed by peptidomics, or liquid chromatography followed by mass spectrometry. In a further embodiment, said method is characterized in that a first mobile phase solvent (mobile phase A solvent) and a second mobile phase solvent (mobile phase B solvent) are used during liquid chromatography; in particular during liquid chromatography in gradient mode. In a further embodiment, the first and second mobile phase solvents are characterized in that they comprise acidified water-acetonitrile-DMSO mixtures, typically acidified with formic acid or other liquid chromatography-compatible known acid in a reversed-phase system. As an example, and in a particular embodiment, the first mobile phase solvent is a solvent comprising 80% water-acetonitrile-DMSO (93-2-5% V/V). In another exemplary embodiment, the second mobile phase solvent is a solvent comprising 20% water-acetonitrile-DMSO (2-93-5% V/V). In a further aspect, said method can be used in the Hydrophilic Interaction Liquid Chromatography (HILIC) mode, for example on an amide column. In said context, the first mobile phase solvent is for example a solvent comprising 40% water-acetonitrile-DMSO (93-2-5% V/V) and the second mobile phase solvent is for example a solvent comprising 60% water-acetonitrile-DMSO (2-93-5% V/V).

The present invention can also be described by the following detailed aspects and relates to a process for the preparation of an anti-adsorption solution, said process comprising the following steps:

1) preparation of a solution of 25 ng/ml to 1 g/ml of one or more proteins and/or peptides in water optionally supplemented with 0.1% to 10% of a first acid;
2) dilution of the solution of step 1 to a concentration of 2.5 ng/ml to 0.1 g/ml of one or more proteins and/or peptides by the addition of a 0.1% to 99% V/V organic solvent solution, said organic solvent solution optionally comprising 0.1% to 10% (m/V) of a second acid;
3) optionally heating the solution obtained in step 2;
4) cooling down the solution obtained step 2 or 3;
5) separation and isolation of the solution obtained in step 4 from any precipitate formed during steps 1 to 4; and
6) dilution of the solution obtained in step 5 in water optionally supplemented with 0.1% to 10% (m/V) of a third acid, thereby obtaining the anti-adsorption solution.

In a further aspect, the process according to the invention comprises the following steps:

1) preparation of a solution of 25 ng/ml to 1 g/ml of one or more proteins and/or peptides in water supplemented with 0.1% to 10% of a first acid;
2) dilution of the solution of step 1 to a concentration of 2.5 ng/ml to 0.1 g/ml of one or more proteins and/or peptides by the addition of a 0.1% to 99% V/V organic solvent solution comprising 0.1% to 10% (m/V) of a second acid;
3) optionally heating the solution obtained in step 2;
4) cooling down the solution obtained step 2 or 3;
5) separation and isolation of the solution obtained in step 4 from any precipitate formed during steps 1 to 4; and
6) dilution of the solution obtained in step 5 in water supplemented with a 0.1% to 10% (m/V) of a third acid, thereby obtaining the anti-adsorption solution.

In a further embodiment, in step 1 of the process a solution of 25 ng/ml to 1 g/ml of one or more proteins and/or peptides in water optionally supplemented with 0.1% to 10% m/V of the first acid is prepared. In still another further embodiment, in step 1 a solution of 25 μg/ml to 25 mg/ml of one or more proteins and/or peptides in water optionally supplemented with 0.1% to 1% m/V of the first acid is prepared.

In a further aspect of the process, the solution prepared in step 1 is diluted to a concentration of 2.5 ng/ml to 0.1 g/ml of one or more proteins and/or peptides by the addition of a 0.1% to 99% V/V organic solvent solution with 0.1% to 10% (m/V) of the second acid. In another embodiment, the solution prepared in step 1 is diluted to a concentration of 2.5 μg/ml to 2.5 mg/ml of one or more proteins and/or peptides by the addition of 0.1%% to 95% (V/V) organic solvent solution with 0.1% to 1% (m/V) of the second acid.

The process according to the invention may optionally comprises a heating step 3. In particular, in said step 3 the solution obtained in step 2 is heated for 1 to 1000 minutes to 30° C. to 120° C.; preferably for 1 to 10 minutes to 75° C. to 100° C.

The process according to the invention further comprises a cooling step 4. In said step 4 the solution is cooled down for 0 to 600 minutes to 40° C. to −35° C.; preferably for 5 to 60 minutes to 8° C. to −8° C.

The process according to the invention comprises a step 5 in which any precipitated formed during steps 1 to 4 is separated and isolated of the solution. Preferably, said step 5 is performed by centrifugation, filtration, or other state-of-the-art separation techniques; preferably by centrifugation. Even more preferably, it is performed by centrifugation for 1 to 600 minutes at 100 g to 100000 g; in particular for 1 to 60 minutes at 1000 g to 50000 g.

In step 6 of the process according to the invention, the solution obtained in step 5 is diluted in water optionally supplemented with a third acid; in particular in water supplemented with 0.1% to 10% (m/V) of a third acid; preferably in water supplemented with 0.% to 1% (m/V) of a third acid.

In a further aspect, the solution obtained in step 5 is diluted in water optionally supplemented with said third acid by the addition of 0.1 to 9.9 parts of the solution obtained in step 4 or 5 in 9.9 to 0.1 parts water optionally supplemented with said third acid.

In the process of the present invention an anti-adsorption solution is prepared starting from a solution of one or more proteins and/or peptides. Said one or more proteins and/or peptides are preferably selected from albumin, ovalbumin, lactalbumin, globulin, gelatin, or a combination thereof. In a preferred embodiment, the one or more proteins is albumin; preferably serum albumin; even more preferably bovine serum albumin.

In a specific exemplified embodiment of the invention, a process is disclosed comprising the following steps:

1) preparation of a solution of 25 μg/ml to 25 mg/ml serum albumin in water supplemented with 0.1% to 1% (m/V) of a first acid;
2) dilution of the solution of step 1 to a concentration of 2.5 μg/ml to 2.5 mg/ml serum albumin by the addition of 0.1% to 95% (V/V) of an organic solvent solution, said organic solvent solution comprising 0.1% to 1% (m/V) of a second acid;
3) optionally heating the solution obtained in step 2 for 1 to 10 minutes to 75° C. to 100° C.;
4) cooling down the solution obtained in step 2 or 3 for 5 to 60 minutes to 8° C. to −8° C.;
5) separation and isolation of the solution obtained in step 4 from any precipitate formed during steps 1 to 4 by centrifugation; and
6) dilution of the solution obtained in step 5 in water supplemented with 0.1% to 1% mV of a third acid, thereby obtaining the anti-adsorption solution.

In a further aspect, the solution obtained in step 4 or 5 is diluted in water supplemented with said third acid by the addition of 0.1 to 9.9 parts of the solution obtained in step 4 or 5 in 9.9 to 0.1 parts water supplemented with said third acid.

In another embodiment, the first acid, the second acid and the third acid are each independently selected from formic acid, acetic acid, trifluoroacetic acid or any other acid. In a specific embodiment, the first acid, the second acid and the third acid are the same; even more specifically, the first acid, the second acid and the third acid are formic acid.

The organic solvent that is used in the process of the present application is further selected from acetonitrile, methanol, isopropanol, denatured ethanol, or any other solvent.

Another aspect of the present invention discloses an anti-adsorption solution obtained by the process according to any of the disclosed embodiments. Also an anti-adsorption powder obtained by drying or freeze-drying said anti-adsorption solution is disclosed. Even further, said anti-adsorption powder is in combination with a suitable solvent or excipient (e.g. a preservative).

The present invention is further directed to the use of an anti-adsorption solution or an anti-adsorption powder as described herein for coating of a recipient or column. In particular, said recipient or column is made of glass or plastic. In another aspect, the use of an anti-adsorption solution or anti-adsorption powder as described herein as a diluent solution for storage, detection, identification and/or separation of peptides; preferably mainly hydrophilic peptides, is described. In another aspect, the use of an anti-adsorption solution or anti-adsorption powder as disclosed herein in peptide analytical methods is described. Said peptide analytical methods are selected from immune-based protein analysis methods, such as ELISA, western blotting, liquid chromatography; mass spectrometry; or peptidomics; preferably wherein the peptide analytical methods are selected from liquid chromatography, mass spectrometry and peptidomics.

In said uses, the anti-adsorption solution can further be combined with a solvent, in particular an organic solvent.

In another aspect, a method for the separation and/or detection of peptides e.g. in a sample is disclosed, wherein the peptides/sample are/is added to or diluted in an anti-adsorption solution as described herein, followed by liquid chromatography optionally followed by mass spectrometry. Preferably, a first mobile phase solvent (mobile phase A solvent) and a second mobile phase solvent (mobile phase B solvent) are used in said method. Further, the liquid chromatography can be performed in gradient mode or in HILIC mode. In still another embodiment, the first mobile phase solvent and the second mobile phase solvent in said method comprise a mixture of acetonitrile and DMSO; said mixture preferably supplemented with water, in particular supplemented with acidified water. In an even more preferred embodiment, the water is acidified with formic acid or any other liquid chromatography-compatible known acid. Said method is further performed in a reversed-phase system. In another embodiment, the first mobile phase solvent in said method is a solvent comprising an 80% water-acetonitrile-DMSO solution (93-2-5% V/V). In still another embodiment, the second mobile phase solvent in said method is a solvent comprising a 20% water-acetonitrile-DMSO solution (2-93-5% V/V). In still another embodiment, the peptides in said method are hydrophilic peptides.

The process and anti-adsorption solution, also referred herein as anti-adsorption diluent according to the present invention is now further described using the following examples.

### Examples

In the examples, anti adsorption diluent and anti adsorption solution are both used, but are both referring to the anti adsorption solution according to an embodiment of the present invention.

### Materials and Reagents

Peptides (amino acid sequences see Table 1) were obtained following customized peptide synthesis from GL Biochem (Shanghai, China) or China Peptides (Shanghai, China). Acetonitrile (LC-MS grade), formic acid and trifluoroacetic acid (both LC-MS grade), methanol (LC-MS grade), isopropanol (LC-MS grade) and dimethylsulfoxide (DMSO) (gas chromatography headspace grade) were all obtained from Biosolve (Valkenswaard, The Netherlands). LC-MS grade water was prepared in-house with the Arium Pro VF TOC purification system (Sartorius, Gottingen, Germany) yielding ≥18.2 MΩcm and ≤5 ppb total organic carbon quality water or commercially purchased from Biosolve (LC-MS grade). Protein LoBind centrifugation tubes were obtained from Eppendorf (Hamburg, Germany). (Ultra) high performance liquid chromatography ((U)HPLC) glass vials with preslit silicon septum (product code: 186000307C, Milford, Mass., USA) and (U)HPLC vial inserts (product code: WAT094171, Milford, Mass., USA) were purchased from Waters. Polypropylene vials with insert (product code: 90273) were purchased from Grace Drive (Columbia, Md., USA). Denatured ethanol (denatured with 1% V/V isopropanol and 1% V/V methylethylketone (MEK)+1,5-Diazabicyclo[4.3.0]non-5-ene (DBN)) (Disolol®) was purchased from Chemlab Analytical (Zedelgem, Belgium).

TABLE 1
Peptide 1-letter amino acid sequence.

| | Peptide | |
|---|---|---|
| Amino acid sequence | ID | SEQ ID No |
| Pilot experiment | | |
| MFPTIPLSRLFDNAMLRAH | | SEQ ID No: 1 |
| Proof of concept experiment | | |
| (pivotal experiment) | | |
| AGTKPQGKPASNLVECVFSLFKKCN | Q11 | SEQ ID No: 2 |
| DIRHRINNSIWRDIFLKRK | Q28 | SEQ ID No: 3 |
| DLRGVPNPWGWIFGR | Q30 | SEQ ID No: 4 |
| DLRNIFLKIKFKKK | Q31 | SEQ ID No: 5 |
| EMRISRIILDFLFLRKK | Q45 | SEQ ID No: 6 |
| EMRKSNNNFFHFLRRI | Q46 | SEQ ID No: 7 |
| EMRLPKILRDFIFPRKK | Q47 | SEQ ID No: 8 |
| ESRLPKILLDFLFLRKK | Q53 | SEQ ID No: 9 |
| ESRLPKIRFDFIFPRKK | Q54 | SEQ ID No: 10 |
| DSRIRMGFDFSKLFGK | Q58 | SEQ ID No: 11 |
| GLWEDILYSLNIIKHNNTKGLHHPIQL | Q101 | SEQ ID No: 12 |
| GLWEDLLYNINRYAHYIT | Q102 | SEQ ID No: 13 |
| SGSLSTFFRLFNRSFTQA | Q171 | SEQ ID No: 14 |
| SGSLSTFFRLFNRSFTQALGK | Q176 | SEQ ID No: 15 |
| NNWNN | Q19 | SEQ ID No: 16 |
| NWN | Q155 | SEQ ID No: 17 |
| AIFILAS | Q13 | SEQ ID No: 18 |
| AITLIFI | Q14 | SEQ ID No: 19 |
| ALILTLVS | Q17 | SEQ ID No: 20 |
| LFSLVLAG | Q132 | SEQ ID No: 21 |
| LFVVTLVG | Q133 | SEQ ID No: 22 |
| LVTLVFV | Q137 | SEQ ID No: 23 |
| SIFTLVA | Q184 | SEQ ID No: 24 |
| VAVLVLGA | Q210 | SEQ ID No: 25 |
| EQLSFTSIGILQLLTIGTRSCWFFYCRY | Q49 | SEQ ID No: 26 |
| KSSAYSLQMGATAIKQVKKLFKKWGW | Q125 | SEQ ID No: 27 |
| MAGNSSNFIHKIKQIFTHR | Q138 | SEQ ID No: 28 |
| TNRNYGKPNKDIGTCIWSGFRHC | Q208 | SEQ ID No: 29 |
| SYPGWSW | Q206 | SEQ ID No: 30 |
| DRVGA | Q34 | SEQ ID No: 31 |
| ERGMT | Q50 | SEQ ID No: 32 |
| ERPVG | Q52 | SEQ ID No: 33 |
| QKGMY | Q160 | SEQ ID No: 34 |
| QRGMI | Q162 | SEQ ID No: 35 |
| SRNAT | Q192 | SEQ ID No: 36 |
| SRNVT | Q193 | SEQ ID No: 37 |

### Anti-Adsorption Diluent Preparation and Vial Coating

Bovine serum albumin (final concentration 10 mg/mL) was dissolved in water acidified with formic acid (final concentration 0.1% m/V). This solution was diluted to a concentration of 2.5 mg/mL bovine serum albumin with acetonitrile acidified with 0.1% m/V formic acid and heated for 5 min at 95° C. with subsequent cooling for 30 min on ice and centrifugation for 20 min, 4° C. at 20000 g. The obtained clear supernatant was diluted by adding 2 parts of the supernatant to 1 part of water acidified with 0.1% m/V formic acid to obtain the anti-adsorption diluent.

During the experiment, certain ultrahigh performance liquid chromatography (UHPLC) vials and inserts were coated by being completely filled with the aforementioned anti-adsorption diluent. The vials were capped and vortexed and incubated for 2 hours at room temperature, emptied after the 2 hour incubation period and tapped dry on a paper towel.

The anti-adsorption diluent was prepared from Bovine Serum Albumin originating from either Merck (product code: 1.12018.0100), referred to as Source 1, Sigma-Aldrich (product code: A3803), referred to as Source 2, and Sigma-Aldrich (product code A9647), referred to as Source 3. The specific source is mentioned throughout the examples wherever applicable. Ovalbumin and lactalbumin were also purchased from Sigma-Aldrich.

### Proof of Principle (Pilot Experiment)

The peptide with amino acid sequence MFPTIPLSRLFDNAMLRAH (SEQ ID No: 1) was stored in uncoated (U)HPLC vial inserts at a concentration of 100 ng/mL in 2 different solvents for a total of approximately 2 h. Solvent 1 consisted of water/acetonitrile (50/50) (V/V)+0.1% (m/V) formic acid; solvent 2 consisted of anti-adsorption diluent, hence meeting the exact organic/inorganic solvent composition of solvent 1. The vials were stored in the UHPLC autosampler compartment during analysis and at 0 min a vial containing solvent 1 and 100 ng/mL peptide was injected onto the UHPLC-MS/MS to serve as reference. The peptide recovery was calculated by dividing the peptide peak area of each time point and condition by the peptide peak area of sample 0 min in solvent 1. Appropriate blank samples were first subjected to the UHPLC-MS/MS for evaluation of interfering substances. This experiment was performed in uniplicate.

The 3 h peptide stability was evaluated by high performance liquid chromatography equipped with a photo diode array detector.

UHPLC-MS/MS of peptide MFPTIPLSRLFDNAMLRAH was conducted with an Acquity H-class quaternary solvent manager, connected to an Acquity Xevo TQ-S triple quadrupole mass spectrometer (Waters, Milford, Mass., USA). The column compartment was kept at 45° C. and the sample compartment at 20° C. Mobile phase A consisted of water/acetonitrile/dimethylsulfoxide 90/5/5 V/V/V supplemented with 0.1% m/V formic acid and mobile phase B consisted of water/acetonitrile/dimethylsulfoxide 5/90/5 V/V/V supplemented with 0.1% m/V formic acid. Chromatography was performed with an Acquity UHPLC BEH300 C₁₈ column (2.1×100 mm, 1.7 μm particle size) (Waters) protected with a suitable guard column. An isocratic period was maintained during 1.5 min at 100% mobile phase A, followed by a linear decrease to 40% mobile phase A at 6.5 min. From 6.5 min to 7.0 min, the fraction of mobile phase A was lowered to 0%, maintained during 1 min (8.0 min time point) and followed by returning to starting conditions after 0.5 min. The starting conditions were maintained until 15.0 min prior to starting a new analytical run. The needle wash (during 6 sec post-injection) consisted of water/acetonitrile/dimethylsulfoxide (45/45/10) (V/V/V) supplemented with 0.1% (m/V) formic acid; methanol was applied as purge solvent and a 90/10 water/methanol (V/V) mixture as seal wash. The mass spectroscopic settings are provided in Table 2. Per analytical run, 2 μL was injected onto the column. Anti-adsorption diluent Source 1 was used during this experiment.

TABLE 2
Mass spectrometer settings for
proof of principle (pilot experiment).
Mass spectrometer settings

| Peptide | MFPTIPLSRLFDNAMLRAH |
|---|---|
| | (SEQ ID No: 1) |
| Capillary voltage (kV) | 3.0 |
| Cone voltage (V) | 32 |
| Source offset (V) | 60 |
| Source temperature | 500 |
| (° C.) | |
| Desolvation gas | 1000 |
| flow (l/h) | |
| Desolvation | 500 |
| temperature (° C.) | |
| Cone gas flow (l/h) | 150 |
| Collision gas flow | 0.16 |
| (ml/min) | |
| Nebuliser (Bar) | 7.0 |
| Collision energy (eV) | 20 |
| Probe position | |
| Vernier probe adjuster: | 5.3 |
| Vertical probe adjuster: | 0.5 |
| Divert valve | 0 → 4.50 min and |
| | 6.51 → 15 min |
| | divert valve to waste |
| | 4.51 → 6.50 min |
| | divert valve to MS |
| MRM | 744.32 > 651.44 |
| MRM timings (min) | 5.4 → 6.2 |

### Peptide Stability

The peptide stability of peptide MFPTIPLSRLFDNAMLRAH (SEQ ID No: 1) was evaluated with HPLC-PDA and the Vydac Everest C₁₈ column. The analytical solution (1 mg/mL) in acetonitrile/water (V/V)+0.1% m/V formic acid (i.e. matching solvent composition as the anti-adsorption diluent without hydrolysate components) was chromatographed with the gradient provided in Table 3. Analysis was performed with a Waters Alliance 2695 HPLC with a Waters 2695 Separations Module, combined with a Flow Through Needle, and a Waters 2996 Photodiode Array Detector with Empower 2 software for data acquisition (Waters, Milford, Mass., USA).

TABLE 3
Analytical HPLC-UV method for peptide stability of peptide
MFPTIPLSRLFDNAMLRAH (SEQ ID No: 1).

| Column + guard | Vydac Everest C₁₈, 4.6 x 250 mm, 5 μm with suitable | | |
|---|---|---|---|
| column | guard column (Grace Drive (Columbia, MD, USA)). | | |
| Flow rate | 1 ml/min | | |
| Injection volume | 20 μL | | |
| Oven temperature | 40° C. | | |
| Sample temperature | 20° C. | | |
| Mobile phase | A: 0.1% (V/V) | | B: 0.1% (V/V) |
| | trifluoroacetic acid | | trifluoroacetic acid in |
| | in 95/5 (V/V) | | 95/5 (V/V) |
| | H₂O/acetonitrile | | acetonitrile/H₂O |
| | Time | Mobile phase A | Mobile phase B |
| | (min) | (%) | (%) |
| Gradient program | 0 | 100 | 0 |
| | 2 | 100 | 0 |
| | 32 | 40 | 60 |
| | 33.5 | 0 | 100 |
| | 38.5 | 0 | 100 |
| | 40 | 100 | 0 |
| | 50 | 100 | 0 |

| | |
|---|---|
| PDA detection | PDA 190-400 nm; quantification at 210 nm |

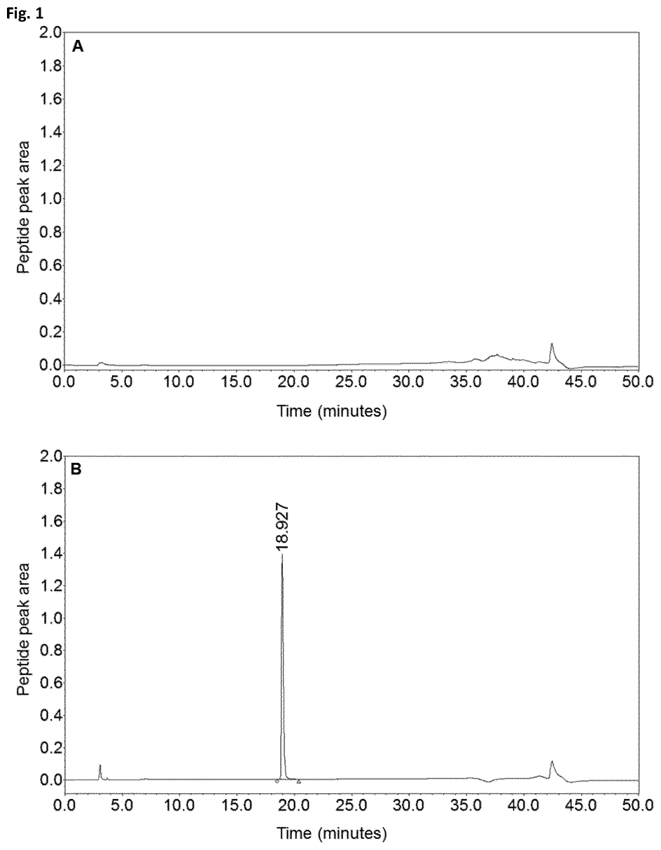

The peptide was injected at 0 h and 3 h. No additional peaks (0.5% reporting threshold) emerged after the 3 h incubation period at 20° C. A recovery (3 h/0 h×100) of 101% was observed (see FIG. 1).

### Proof of Concept (Pivotal Experiment).

36 model peptides were selected for evaluation of the anti-adsorption diluent general functionality. The most suitable solvent composition and need of vial coating with the anti-adsorption diluent was evaluated via a D-optimal design model generated by Modde software (Umetrics, Umea, Sweden). The design is provided in Table 4.

TABLE 4
D-optimal design (proof of concept pivotal experiment).

| | | | Fractions | | | |
|---|---|---|---|---|---|---|
| | Analysis | | | Formic acid 10% | | Anti-adsorption |
| Experiment | order | Vial | Acetonitrile | m/V in water | Water | solution |
| 1 | 27 | uncoated vial | 0.75 | 0.25 | 0.00 | 0.00 |
| 2 | 30 | uncoated vial | 0.00 | 1.00 | 0.00 | 0.00 |
| 3 | 11 | uncoated vial | 0.00 | 0.25 | 0.50 | 0.25 |
| 4 | 10 | uncoated vial | 0.00 | 0.50 | 0.00 | 0.50 |
| 5 | 9 | uncoated vial | 0.00 | 0.75 | 0.25 | 0.00 |
| 6 | 21 | uncoated vial | 0.25 | 0.25 | 0.00 | 0.50 |
| 7 | 22 | uncoated vial | 0.25 | 0.25 | 0.50 | 0.00 |
| 8 | 26 | uncoated vial | 0.50 | 0.50 | 0.00 | 0.00 |
| 9 | 31 | coated vial | 0.00 | 0.25 | 0.75 | 0.00 |
| 10 | 18 | coated vial | 0.00 | 0.25 | 0.00 | 0.75 |
| 11 | 24 | coated vial | 0.00 | 0.25 | 0.25 | 0.50 |
| 12 | 23 | coated vial | 0.00 | 0.75 | 0.00 | 0.25 |
| 13 | 14 | coated vial | 0.00 | 0.50 | 0.50 | 0.00 |
| 14 | 37 | coated vial | 0.50 | 0.25 | 0.00 | 0.25 |
| 15 | 17 | coated vial | 0.50 | 0.25 | 0.25 | 0.00 |
| 16 | 13 | coated vial | 0.25 | 0.75 | 0.00 | 0.00 |
| 17 | 15 | coated vial | 0.19 | 0.44 | 0.19 | 0.19 |
| 18 | 38 | coated vial | 0.19 | 0.44 | 0.19 | 0.19 |
| 19 | 19 | coated vial | 0.19 | 0.44 | 0.19 | 0.19 |
| 20 | 4 | uncoated vial | 0.75 | 0.25 | 0.00 | 0.00 |
| 21 | 29 | uncoated vial | 0.00 | 1.00 | 0.00 | 0.00 |
| 22 | 12 | uncoated vial | 0.00 | 0.25 | 0.50 | 0.25 |
| 23 | 7 | uncoated vial | 0.00 | 0.50 | 0.00 | 0.50 |
| 24 | 2 | uncoated vial | 0.00 | 0.75 | 0.25 | 0.00 |
| 25 | 8 | uncoated vial | 0.25 | 0.25 | 0.00 | 0.50 |
| 26 | 34 | uncoated vial | 0.25 | 0.25 | 0.50 | 0.00 |
| 27 | 1 | uncoated vial | 0.50 | 0.50 | 0.00 | 0.00 |
| 28 | 28 | coated vial | 0.00 | 0.25 | 0.75 | 0.00 |
| 29 | 5 | coated vial | 0.00 | 0.25 | 0.00 | 0.75 |
| 30 | 16 | coated vial | 0.00 | 0.25 | 0.25 | 0.50 |
| 31 | 35 | coated vial | 0.00 | 0.75 | 0.00 | 0.25 |
| 32 | 6 | coated vial | 0.00 | 0.50 | 0.50 | 0.00 |
| 33 | 25 | coated vial | 0.50 | 0.25 | 0.00 | 0.25 |
| 34 | 33 | coated vial | 0.50 | 0.25 | 0.25 | 0.00 |
| 35 | 3 | coated vial | 0.25 | 0.75 | 0.00 | 0.00 |
| 36 | 32 | coated vial | 0.19 | 0.44 | 0.19 | 0.19 |
| 37 | 20 | coated vial | 0.19 | 0.44 | 0.19 | 0.19 |
| 38 | 36 | coated vial | 0.19 | 0.44 | 0.19 | 0.19 |

The peptides were analyzed at the concentration specified in Table 5. For model optimization, the analysis wizard was used. In brief, the data were first visually controlled for outliers using the replicate plot; secondly, the data were transformed whenever deemed necessary (e.g. logarithmic transformation) when the histogram demonstrated this necessity. In the next step, the non-significant model terms (i.e. those not contributing to a higher R² and/or Q²) were removed via the coefficient plot. Outliers in the residuals normal probability plot and the observed versus predicted plot were removed. The optimum was calculated using the optimizer tool of the Modde software.

TABLE 5
Peptide concentration pivotal (proof of concept) experiment.

| Peptide amino acid | Concentration | Peptide amino acid | Concentration |
|---|---|---|---|
| sequence (Peptide ID) | (nM) | sequence (Peptide ID) | (nM) |
| AGTKPQGKPASNLVECVFSLFKKC | 0.5 | NWN (Q19) | 0.05 |
| N (Q11) | | | |
| DIRHRINNSIWRDIFLKRK (Q28) | 0.5 | AIFILAS (Q13) | 0.1 |
| DLRGVPNPWGWIFGR (Q30) | 0.5 | AITLIFI (Q14) | 0.5 |
| DLRNIFLKIKFKKK (Q31) | 1 | ALILTLVS (Q17) | 0.5 |
| EMRISRIILDFLFLRKK (Q45) | 1 | LFSLVLAG (Q132) | 10 |
| EMRKSNNNFFHFLRRI (Q46) | 0.5 | LFVVTLVG (Q133) | 1 |
| EMRLPKILRDFIFPRKK (Q47) | 0.1 | LVTLVFV (Q137) | 0.5 |
| ESRLPKILLDFLFLRKK (Q53) | 1 | SIFTLVA (Q184) | 1 |
| ESRLPKIRFDFIFPRKK (Q54) | 0.1 | VAVLVLGA (Q210) | 0.5 |
| QRGMI (Q162) | 0.05 | EQLSFTSIGILQLLTIGTRSCWFFY | 10 |
| | | CRY (Q49) | |
| DSRIRMGFDFSKLFGK (Q58) | 0.5 | KSSAYSLQMGATAIKQVKKLFKKW | 0.5 |
| | | GW (Q125) | |
| SRNVT(Q193) | 0.5 | MAGNSSNFIHKIKQIFTHR (Q138) | 0.1 |
| GLWEDILYSLNIIKHNNTKGLHHPI | 1 | TNRNYGKPNKDIGTCIWSGFRHC | 0.5 |
| QL (Q101) | | (Q208) | |
| GLWEDLLYNINRYAHYIT (Q102) | 10 | SYPGWSW (Q206) | 0.025 |
| SGSLSTFFRLFNRSFTQA (Q171) | 1 | DRVGA (Q34) | 0.5 |
| SGSLSTFFRLFNRSFTQALGK | 0.05 | ERGMT (Q50) | 0.025 |
| (Q176) | | | |
| NNWNN (Q19) | 0.05 | ERPVG (Q52) | 0.1 |
| SRNAT (Q192) | 0.025 | QKGMY (Q160) | 1 |

Using the same UHPLC-MS/MS apparatus as previously described in the proof of principle, the column compartment was kept at 60° C. and the sample compartment at 10° C. respectively. An Acquity UHPLC BEH300 C₁₈ (2.1×100 mm; 1.7 μm) column equipped with a suitable guard column or an Acquity UHPLC BEH Amide (2.1×100 mm; 1.7 μm) column equipped with a suitable guard column were employed for chromatographic separation and were both obtained from Waters (Milford, Mass., USA). Mobile phase A consisted of water/acetonitrile/dimethylsulfoxide 93/2/5 (V/V/V) supplemented with 0.1% (m/V) formic acid, while mobile phase B consisted of water/acetonitrile/dimethylsulfoxide 2/93/5 (V/V/V) supplemented with 0.1% (m/V) formic acid. The gradient composition for both column systems is given in Table 6. Per analytical run, 10 μL of the analytical sample solution was injected onto the column. The peptides analyzed using the HILIC amide or C₁₈ column system are mentioned in Table 7. Anti-adsorption diluent Source 1 was used during this experiment.

TABLE 6
Gradient solvent composition.

| | | Solvent composition (%) | | | |
|---|---|---|---|---|---|
| | | C18 column | | HILIC amide | |
| | | system | | column system | |
| | Flow | Mobile | Mobile | Mobile | Mobile |
| Time | rate | phase | phase | phase | phase |
| (min) | (mL/min) | A | B | A | B |
| 0.00 | | 100 | 0 | 0 | 100 |
| 2.00 | | 100 | 0 | 0 | 100 |
| 9.00 | | 40 | 60 | 60 | 40 |
| 9.50 | 0.5 | 14.2 | 85.8 | 85.8 | 14.2 |
| 11.00 | | 14.2 | 85.8 | 85.8 | 14.2 |
| 11.01 | | 100 | 0 | 0 | 100 |
| 15.00 | | 100 | 0 | 0 | 100 |

TABLE 7
Peptides of proof of concept, according to the
chromatographic C₁₈ and HILIC amide system.

| | HILIC amide |
|---|---|
| C₁₈ column system | column system |
| AGTKPQGKPASNLVECVFSLFKKCN (Q11) | NNWNN (Q19) |
| DIRHRINNSIWRDIFLKRK (Q28) | NWN (Q155) |
| DLRGVPNPWGWIFGR (Q30) | DRVGA (Q34) |
| DLRNIFLKIKFKKK (Q31) | ERGMT (Q50) |
| EMRISRIILDFLFLRKK (Q45) | ERPVG (Q52) |
| EMRKSNNNFFHFLRRI (Q46) | QKGMY (Q160) |
| EMRLPKILRDFIFPRKK (Q47) | QRGMI (Q162) |
| ESRLPKILLDFLFLRKK (Q53) | SRNVT (Q193) |
| ESRLPKIRFDFIFPRKK (Q54) | |
| DSRIRMGFDFSKLFGK (Q58) | |
| GLWEDILYSLNIIKHNNTKGLHHPIQL (Q101) | |
| GLWEDLLYNINRYAHYIT (Q102) | |
| SGSLSTFFRLFNRSFTQA (Q171) | |
| SGSLSTFFRLFNRSFTQALGK (Q176) | |
| AIFILAS (Q13) | |
| AITLIFI (Q14) | |
| ALILTLVS (Q17) | |
| LFSLVLAG (Q132) | |
| LFVVTLVG (Q133) | |
| LVTLVFV (Q137) | |
| SIFTLVA (Q184) | |
| VAVLVLGA (Q210) | |
| EQLSFTSIGILQLLTIGTRSCWFFYCRY (Q49) | |
| KSSAYSLQMGATAIKQVKKLFKKWGW (Q125) | |
| MAGNSSNFIHKIKQIFTHR (Q138) | |
| TNRNYGKPNKDIGTCIWSGFRHC (Q208) | |
| SYPGWSW (Q206) | |
| SRNAT (Q192) | |

The mass spectrometer settings are provided in Tables 8 and 9.

TABLE 8
Mass spectrometer settings for proof of concept (pivotal experiment).
Mass spectrometer settings

| | |
|---|---|
| Source offset | 50.0 V |
| Capillary voltage | 3.00 kV |
| Cone voltage | 30.00 V |
| Source temperature | 150° C. |
| Desolvation gas flow | 1000 l/h |
| Desolvation temperature | 500° C. |
| Collision energy MS1 | 4 eV |
| Cone gas flow | 150 l/h |
| Collision gas flow | 0.16 ml/min |
| Nebuliser gas flow | 7.00 bar |
| Probe position | |
| Vernier probe adjuster: | 5.20 |
| Vertical probe adjuster: | 0.7 mm |
| LM resolution 1: | 2.5 |
| HM resolution 1: | 14.9 |
| Ion energy: | 0.1 |
| LM resolution 2: | 2.8 |
| HM resolution 2: | 14.9 |
| Ion energy: | 0.8 |
| Divert valve | 0.00 min-2.00 min Waste |
| | 2.01 min-9.30 min LC |
| | 9.31 min-15.00 min Waste |

The peptide MRM's are provided in Table 9. Per peptide, a quantifier and qualifier were monitored simultaneously. To minimize overlap of the different peptides and hence reducing sensitivity, the peptides were divided over different analytical runs.

TABLE 9
Peptide specific MS and MS/MS settings.

| | Qualifier | | | | | |
|---|---|---|---|---|---|---|
| Peptide amino | (qual)/ | Collision | | | Time | |
| acid sequence | Quantifier | Energy | Precursor | Fragment | frame | Analytical |
| (Peptide ID) | (quant) | (eV) | ion | ion | (min) | run |
| AGTKPQGKPASNLVECVFS | Quant | 22.00 | 667.25 | 662.69 | 7.47-15.00 | Run 2 C₁₈ |
| LFKKCN(Q11) | Qual | 22.00 | 667.25 | 949.76 | | |
| DIRHRINNSIWRDIFLKRK | Quant and | 20.00 | 621.20 | 589.05 | 0.00-5.85 | Run 3 C₁₈ |
| (Q28) | qual | | | | | |
| DLRGVPNPWGWIFGR | Quant and | 35.00 | 886.82 | 828.96 | 7.41-7.81 | Run 1 C₁₈ |
| (Q30) | qual | | | | | |
| DLRNIFLKIKFKKK | Quant | 30.00 | 598.99 | 499.25 | 0.00-5.99 | Run 2 C₁₈ |
| (Q31) | Qual | 30.00 | 598.99 | 612.32 | | |
| EMRISRIILDFLFLRKK | Quant | 20.00 | 546.50 | 615.31 | 7.02-7.42 | Run 1 C₁₈ |
| (Q45) | Qual | | 546.50 | 688.78 | | |
| EMRKSNNNFFHFLRRI | Quant | 20.00 | 528.90 | 495.75 | 0.00-6.01 | Run 1 C₁₈ |
| (Q46) | Qual | | 528.90 | 625.99 | | |
| EMRLPKILRDFIFPRKK | Quant | 20.00 | 548.70 | 543.85 | 6.27-6.67 | Run 1 C₁₈ |
| (Q47) | Qual | | 548.70 | 687.91 | | |
| ESRLPKILLDFLFLRKK | Quant | 30.00 | 706.29 | 403.59 | 6.92-7.32 | Run 3 C₁₈ |
| (Q53) | Qual | | 706.29 | 1066.47 | | |
| ESRLPKIRFDFIFPRKK | Quant | 20.00 | 545.19 | 621.65 | 5.91-6.31 | Run 2 C₁₈ |
| (Q54) | Qual | | 545.19 | 751.67 | | |
| DSRIRMGFDFSKLFGK | Quant | 30.00 | 635.72 | 539.90 | 6.54-6.94 | Run 3 C₁₈ |
| (Q58) | Qual | | 635.72 | 721.27 | | |
| GLWEDILYSLNIIKHNNTK | Quant | 20.00 | 793.55 | 750.63 | 7.32-7.72 | Run 2 C₁₈ |
| GLHHPIQL (Q101) | Qual | | 793.55 | 1000.52 | | |
| GLWEDLLYNINRYAHYIT | Quant | 35.00 | 1128.08 | 1011.90 | 7.96-8.80 | Run 1 C₁₈ |
| (Q102) | Qual | | 1128.08 | 1068.50 | | |
| SGSLSTFFRLFNRSFTQA | Quant and | 45.00 | 1034.94 | 999.04 | 7.03-7.43 | Run 3 C₁₈ |
| (Q171) | qual | | | | | |
| SGSLSTFFRLFNRSFTQAL | Quant | 30.00 | 789.18 | 1011.06 | 7.06-7.46 | Run 2 C₁₈ |
| GK (Q176) | Qual | | 789.18 | 1067.55 | | |
| NNWNN (Q19) | Quant | 30.00 | 661.20 | 229.04 | 0.00-7.40 | Run 2 HILIC |
| | Qual | | 661.20 | 415.06 | | amide |
| NWN (Q155) | Quant | 20.00 | 433.12 | 273.05 | 0.00-6.80 | Run 1 HILIC |
| | Qual | | 433.12 | 284.00 | | amide |
| AIFILAS (Q13) | Quant | 32.00 | 734.37 | 332.10 | 6.54-7.00 | Run 1 C₁₈ |
| | Qual | | 734.37 | 445.11 | | |
| AITLIFI (Q14) | Quant | 20.00 | 790.37 | 512.16 | 7.59-7.99 | Run 1 C₁₈ |
| | Qual | | 790.37 | 659.18 | | |
| ALILTLVS (Q17) | Quant | 21.00 | 829.43 | 625.21 | 6.88-7.28 | Run 2 C₁₈ |
| | Qual | | 829.43 | 724.24 | | |
| LFSLVLAG (Q132) | Quant | 30.00 | 819.24 | 395.18 | 6.90-7.30 | Run 1 C₁₈ |
| | Qual | | 819.24 | 560.13 | | |
| LFVVTLVG (Q133) | Quant | 22.00 | 847.44 | 673.19 | 6.97-7.37 | Run 2 C₁₈ |
| | Qual | | 847.44 | 772.23 | | |
| LVTLVFV (Q137) | Quant | 33.00 | 790.42 | 314.10 | 7.20-7.60 | Run 2 C₁₈ |
| | Qual | | 790.42 | 409.11 | | |
| SIFTLVA (Q184) | Quant | 30.00 | 750.28 | 431.06 | 6.75-7.15 | Run 3 C₁₈ |
| | Qual | | 750.28 | 544.06 | | |
| VAVLVLGA (Q210) | Quant | 30.00 | 741.40 | 383.12 | 6.38-6.78 | Run 3 C₁₈ |
| | Qual | | 741.40 | 482.16 | | |
| EQLSFTSIGILQLLTIGTR | Quant | 25.00 | 1116.46 | 1371.77 | 8.80-15.00 | Run 1 C₁₈ |
| SCWFFYCRY (Q49) | Qual | | 1116.46 | 1545.75 | | |
| KSSAYSLQMGATAIKQVKK | Quant | 26.00 | 747.32 | 995.52 | 6.40-6.95 | Run 2 C₁₈ |
| LFKKWGW (Q125) | Qual | | 747.32 | 1061.20 | | |
| MAGNSSNFIHKIKQIFTHR | Quant | 27.00 | 744.21 | 1014.60 | 5.80-6.50 | Run 3 C₁₈ |
| (Q138) | Qual | | 744.21 | 1050.00 | | |
| TNRNYGKPNKDIGTCIWSG | Quant | 22.00 | 668.45 | 706.29 | 5.30-6.30 | Run 1 C₁₈ |
| FRHC (Q208) | Qual | | 668.45 | 889.38 | | |
| SYPGWSW (Q206) | Quant and | 25.00 | 882.41 | 632.29 | 6.25-6.80 | Run 2 C₁₈ |
| | qual | | | | | |
| DRVGA (Q34) | Quant | 27.00 | 518.25 | 403.33 | 7.05-7.60 | Run 1 HILIC |
| | Qual | | 518.25 | 447.33 | | amide |
| ERGMT (Q50) | Quant | 29.00 | 593.28 | 492.28 | 7.20-7.70 | Run 1 HILIC |
| | Qual | | 593.28 | 531.35 | | amide |
| ERPVG (Q52) | Quant | 30.00 | 558.33 | 269.27 | 7.00-7.50 | Run 1 HILIC |
| | Qual | | 558.33 | 429.31 | | amide |
| QKGMY (Q160) | Quant | 35.00 | 626.33 | 428.27 | 0.00-7.50 | Run 3 HILIC |
| | Qual | | 626.33 | 608.30 | | amide |
| QRGMI (Q162) | Quant | 35.00 | 604.37 | 308.24 | 6.70-7.30 | Run 1 HILIC |
| | Qual | | 604.37 | 491.30 | | amide |
| SRNAT (Q192) | Quant | 30.00 | 549.28 | 448.31 | 7.50-15.00 | Run 2 HILIC |
| | Qual | | 549.28 | 487.35 | | amide |
| SRNVT (Q193) | Quant | 31.00 | 577.33 | 342.28 | 7.50-15.00 | Run 3 HILIC |
| | Qual | | 577.33 | 476.35 | | amide |

Principal component analysis and hierarchical cluster analysis. Descriptor calculation (Dragon 5.5 (Talete, Milan, Italy), HyperChem 8.0 and Marvin Beans 5.3.3 (Chemaxon, Budapest, Hungary) software), principal component analysis (SIMCA 15 (Sartorius Stedim Biotech, Gottingen, Germany)) and hierarchical cluster analysis (SPSS 25 (IBM, IL, USA)) were carried out as previously described by Wynendaele et al. [18] (included herein by reference).

Composition of the anti-adsorption diluent. Three batches of anti-adsorption diluent Source 1 were prepared as previously described. The anti-adsorption diluents were analyzed using the high-resolution Synapt G2Si (Waters) mass spectrometer (HRMS) equipped with a nanosource (lock spray) operated in positive ion mode in the HDDA-ion mobility mode. Chromatography was performed using nanoAcquity (Waters) equipment with a Acquity UHPLC M-Class HSS T3 column (75 μm×250 mm; 1.8 μm) preceded by a nanoAcquity UHPLC Symmetry C₁₈ trap column (180 μm×20 mm; 100 Å; 5 μm). Onto the trap column, 8 μL of sample was injected and elution was performed during a 60 min run time, starting at 99% mobile phase A (97/3 V/V water/DMSO supplemented with 0.1% m/V formic acid) and 1% mobile phase B (acetonitrile supplemented with 0.1% m/V formic acid) during an initial isocratic phase of 0.1 min. Subsequently, a gradient was applied to obtain 60% mobile phase A at 30 min and 15% mobile phase A at 32 min. Mobile phase A was kept at 15% during 7 min followed by returning to starting conditions at 40 min and kept for the remaining 20 min at this solvent composition. The flow rate was set to 0.3 μL/min during the entire run with a trapping flow rate of 8 μL/min with a 5 min sample loading time (sample loading was performed with 99.5% mobile phase A). Wash solvents consisted of a weak wash solvent (water) and a strong wash solvent (acetonitrile). The column was maintained at 40° C. and the sample compartment at 4° C. The mass spectrometer settings are provided in Table 10.

TABLE 10
Mass spectrometer settings structure elucidation anti-adsorption diluent.
Mass spectrometer settings

| | |
|---|---|
| Capillary (kV) | 2.5 |
| Source Temperature (° C.) | 100 |
| Sampling Cone | 40 |
| Source Offset | 80 |
| Source Gas Flow (mL/min) | 0 |
| Desolvation Temperature (° C.) | 150 |
| Cone Gas Flow (L/Hr) | 50 |
| Nanoflow Gas Pressure (Bar) | 0.3 |
| Purge Gas Flow (ml_/h) | 600 |
| Desolvation Gas Flow (L/Hr) | 600 |
| Nebuliser Gas Flow (Bar) | 6.5 |
| LM Resolution | 4.7 |
| HM Resolution | 15 |
| Aperture 1 | 0 |
| Pre-filter | 2 |
| Ion Energy | 1 |
| Trap Collision Energy | 4 |
| Transfer Collision Energy | 2 |
| Trap Gas Flow (mL/min) | 2 |
| HeliumCellGasFlow | 180 |
| IMS Gas Flow (mL/min) | 90 |
| Detector | 2954 |
| DetectorCache | 2300 |
| LockSpray Capillary (kV) | 4 |
| Collision Energy | 4 |
| Acceleration 1 | 70 |
| Acceleration2 | 200 |
| Aperture2 | 40 |
| Transportl | 70 |
| Transport2 | 70 |
| Steering | 0.5 |
| Tube Lens | 45 |
| Pusher | 1900 |
| Pusher Offset | 0.25 |
| Puller | 1370 |
| Pusher Cycle Time (μs) | Automatic |
| Pusher Width (μs) | Automatic |
| Collector | 50 |
| Collector Pulse | 10 |
| Stopper | 10 |
| Stopper Pulse | 20 |
| Entrance | 62 |
| Static Offset | 180 |
| Puller Offset | 0 |
| Reflectron Grid (kV) | 1.473 |
| Flight Tube (kV) | 10 |
| Reflectron (kV) | 3.78 |
| Trap DC Entrance | 0 |
| Trap DC Bias | 45 |
| Trap DC | 0 |
| Trap DC Exit | 3 |
| IMS DC Entrance | 25 |
| Helium Cell DC | 35 |
| Helium Exit | −5 |
| IMSBias | 3 |
| IMS DC Exit | 0 |
| Transfer DC Entrance | 4 |
| Transfer DC Exit | 15 |
| Trap Wave Velocity (m/s) | 311 |
| Trap Wave Height (V) | 4 |
| IMS Wave Velocity (m/s) | 650 |
| IMS Wave Height (V) | 40 |
| Transfer Wave Velocity (m/s) | 190 |
| Transfer Wave Height (V) | 4 |
| Step Wave 1 In Velocity (m/s) | 300 |
| Step Wave 1 In Height | 10 |
| Step Wave 1 Out Velocity (m/s) | 300 |
| Step Wave 1 Out Height | 0 |
| Step Wave 2 Velocity (m/s) | 300 |
| Step Wave 2 Height | 0 |
| Step Wave TransferOffset | 25 |
| Step Wave DiffAperturel | 3 |
| Step Wave DiffAperture2 | 0 |
| Use Automatic RF Settings | TRUE |
| StepWavel RFOffset | 100 |
| StepWave2RFOffset | 150 |
| Target Enhancement Mode | EDC |
| Target Enhancement Mass | 556 |
| Target Enhancement Trap Height (V) | 12 |
| Target Enhancement Extract Height (V) | 8 |
| Mobility Trapping Release Time (μs) | 500 |
| Mobility Trap Height (V) | 15 |
| Mobility Extract Height (V) | 0 |
| IMS Wave Delay (μs) | 1000 |
| Variable Wave Height Enabled | FALSE |
| Wave Height Ramp Type | Linear |
| Wave Height Start (V) | 10 |
| Wave Height End (V) | 40 |
| Wave Height Ramp (%) | 100 |
| Variable Wave Velocity Enabled | TRUE |
| Wave Velocity Ramp Type | Linear |
| Wave Velocity Start (m/s) | 2500 |
| Wave Velocity End (m/s) | 400 |
| Wave Velocity Using Full IMS | TRUE |
| Wave Velocity Ramp (%) | 100 |
| Wave Velocity Look Up Table | |
| Acquisition mass range | |
| Start mass | 50 |
| End mass | 5000 |
| Calibration mass range | |
| Start mass | 72.132 |
| End mass | 1285.203 |
| ACQUISITION | |
| Survey Start Time | 10 |
| Survey End Time | 55 |
| Switch to MS/MS when | Intensity rising above threshold |
| Intensity Threshold | 3000 |
| Survey Scan Time | 0.2 |
| Survey Interscan Time | 0.01 |
| Survey Data Format | Continuum |
| ADC Sample Frequency (GHz) | 3 |
| MS/MS | |
| MSMS Start Mass | 50 |
| MSMS End Mass | 5000 |
| MSMS to MS Switch Criteria | TIC rising above threshold |
| Switchback threshold | 100000 |
| Trap MSMS Collision Energy Ramp Start (eV) | 30 |
| Trap MSMS Collision Energy Ramp End (eV) | 30 |
| Trap MSMS Collision Energy Ramp Low Mass (Da) | 50 |
| Trap MSMS Collision Energy Ramp High Mass (Da) | 5000 |
| Trap MSMS Collision Energy Ramp LM Start (eV) | 6 |
| Trap MSMS Collision Energy Ramp LM End (eV) | 9 |
| Trap MSMS Collision Energy Ramp HM Start (eV) | 147 |
| Trap MSMS Collision Energy Ramp HM End (eV) | 183 |
| TRANFER COLLISION ENERGY | |
| Using MSMS Auto Trap Collision Energy (eV) | 2 |
| CONE VOLTAGE | |
| Cone (V) | 40 |
| Q-DDA Mode | 0 |
| MSe Low CE | 6 |
| MSe High Start CE | 10 |
| MSe High End CE | 20 |
| Hi MSe Low CE | 6 |
| Hi MSe High Start CE | 10 |
| Hi MSe High End CE | 20 |
| Q-DDA Interval | 10 |
| MS Survey DRE | 99 |

De novo sequencing was performed with PEAKS software [19]. Peptides with an average local confidence (ALC) exceeding 80% were considered as sufficiently identified.

Matrix effects of the anti-adsorption diluent. In brief, the peptide EMRKSNNNFFHFLRRI (Q46; SEQ ID No. 7) was post-column infused whilst anti-adsorption diluent was chromatographed using the aforementioned UHPLC-MS/MS method (see pivotal experiment).

Functional evaluation of anti-adsorption diluent from 3 sources. The functionality of anti-adsorption diluent Source 1, Source 2, and Source 3 was evaluated via the peptide MFPTIPLSRLFDNAMLRAH (SEQ ID No. 1). The peptide was diluted in either solvent (50/50 V/V water/acetonitrile supplemented with 0.1% m/V formic acid) or in 1 of the 3 anti-adsorption diluent sources to a peptide concentration of 100 ng/mL. The solution was either transferred to an insert and immediately analyzed via UHPLC-MS/MS or repeatedly transferred to inserts (5 inserts in total), with analysis of the 5^{th} insert. UHPLC-MS/MS was carried out as previously discussed in the proof of principle (pilot experiment)-section. To prevent peptide degradation, each sample was prepared immediately prior to injection. Per condition 3 replicates were analyzed.

Anti-adsorption diluent applicability for polypropylene inserts. The functionality of the anti-adsorption diluent Source 1 in polypropylene inserts was evaluated via the peptide MFPTIPLSRLFDNAMLRAH (100 ng/mL). The peptide was analyzed in (I) a glass insert with 50/50 V/V water/acetonitrile+0.1% m/V formic acid, (II) a polypropylene insert with 50/50 V/V water/acetonitrile+0.1% m/V formic acid, (III) a glass insert with anti-adsorption diluent Source 1, and (IV) a polypropylene insert with anti-adsorption diluent. The UHPLC-MS/MS method provided in the proof of principle experiment was applied. The experiment was performed in triplicate.

### Anti-Adsorption Diluent (AAD) as a Function of Process Variables

Alternative AAD's were prepared as follows, all AAD's were compared to the AAD as previously described. The differences with the aforementioned process are in bolt.

AAD from Different Protein Sources and/or Other Organic Solvents

Ovalbumin or lactalbumin (final concentration 10 mg/mL) was dissolved in water acidified with formic acid (final concentration 0.1% m/V). This solution was diluted to a concentration of 2.5 mg/mL lactalbumin or ovalbumin with acetonitrile, denatured ethanol, or isopropanol acidified with 0.1% m/V formic acid and heated for 5 min at 95° C. with subsequent cooling for 30 min on ice and centrifugation for 20 min, 4° C. at 20000 g. The obtained clear supernatant was diluted by adding 2 parts of the supernatant to 1 part of water acidified with 0.1% m/V formic acid to obtain the anti-adsorption diluent. Lactalbumin is referred to as LAC, ovalbumin as OVAL, denatured ethanol as EtOH, acetonitrile as ACN, and isopropanol as IPA.

AAD with Trifluoroacetic Acid

Bovine serum albumin (final concentration 10 mg/mL) was dissolved in water acidified with trifluoroacetic acid (final concentration 0.1% m/V). This solution was diluted to a concentration of 2.5 mg/mL bovine serum albumin with acetonitrile acidified with 0.1% m/V trifluoroacetic acid and heated for 5 min at 95° C. with subsequent cooling for 30 min on ice and centrifugation for 20 min, 4° C. at 20000 g. The obtained clear supernatant was diluted by adding 2 parts of the supernatant to 1 part of water acidified with 0.1% m/V fluoroacetic acid to obtain the anti-adsorption diluent referred to as AAD BSA+TFA.

AAD Precipitated with 50/50 V/V Water/Acetonitrile+0.1% m/V Formic Acid

Bovine serum albumin (final concentration 10 mg/mL) was dissolved in water acidified with formic acid (final concentration 0.1% m/V). This solution was diluted to a concentration of 2.5 mg/mL bovine serum albumin with acetonitrile acidified with 0.1% m/V formic acid to obtain a ratio of 50/50 V/V water/acetonitrile+0.1 m/V formic acid (previously 25/75 V/V) and heated for 5 min at 95° C. with subsequent cooling for 30 min on ice and centrifugation for 20 min, 4° C. at 20000 g. The obtained clear supernatant was diluted by adding 2 parts of the supernatant to 1 part of water acidified with 0.1% m/V formic acid to obtain the anti-adsorption diluent referred to as AAD BSA+FA 50/50 V/V ACN/water.

AAD Precipitated with 10/90 V/V Water/Acetonitrile+0.1% m/V Formic Acid

Bovine serum albumin (final concentration 25 mg/mL) was dissolved in water acidified with formic acid (final concentration 0.1% m/V). This solution was diluted to a concentration of 2.5 mg/mL bovine serum albumin with acetonitrile acidified with 0.1% m/V formic acid to obtain a ratio of 10/90 V/V water/acetonitrile+0.1 m/V formic acid (previously 25/75 V/V) and heated for 5 min at 95° C. with subsequent cooling for 30 min on ice and centrifugation for 20 min, 4° C. at 20000 g. The obtained clear supernatant was diluted by adding 2 parts of the supernatant to 1 part of water acidified with 0.1% m/V formic acid to obtain the anti-adsorption diluent referred to as AAD BSA+FA 10/90 V/V ACN/water.

AAD without Formic Acid

Bovine serum albumin (final concentration 10 mg/mL) was dissolved in water. This solution was diluted to a concentration of 2.5 mg/mL bovine serum albumin with acetonitrile and heated for 5 min at 95° C. with subsequent cooling for 30 min on ice and centrifugation for 20 min, 4° C. at 20000 g. The obtained clear supernatant was diluted by adding 2 parts of the supernatant to 1 part of water obtain the anti-adsorption diluent referred to as AAD BSA-FA.

AAD without Heating

Bovine serum albumin (final concentration 10 mg/mL) was dissolved in water acidified with formic acid (final concentration 0.1% m/V). This solution was diluted to a concentration of 2.5 mg/mL bovine serum albumin with acetonitrile acidified with 0.1% m/V formic acid and kept at room temperature for 5 min with subsequent cooling for 30 min on ice and centrifugation for 20 min, 4° C. at 20000 g. The obtained clear supernatant was diluted by adding 2 parts of the supernatant to 1 part of water acidified with 0.1% m/V formic acid to obtain the anti-adsorption diluent referred to as AAD BSA+FA WITHOUT heating.

UHPLC-MS/MS analysis was performed as described in the proof of concept (pivotal experiment)-section. All were analyzed as 6 technical replicates and standardization occurred against the aforementioned AAD (i.e. Bovine serum albumin Source 2 (final concentration 10 mg/mL) was dissolved in water acidified with formic acid (final concentration 0.1% m/V). This solution was diluted to a concentration of 2.5 mg/mL bovine serum albumin with acetonitrile acidified with 0.1% m/V formic acid and heated for 5 min at 95° C. with subsequent cooling for 30 min on ice and centrifugation for 20 min, 4° C. at 20000 g. The obtained clear supernatant was diluted by adding 2 parts of the supernatant to 1 part of water acidified with 0.1% m/V formic acid to obtain the anti-adsorption diluent referred to as control.)

Standardization was obtained by dividing the peptide peak area of each injection by the mean peptide peak area of the control (i.e. mean 100%).

### Results and Discussion

### Proof of Principle

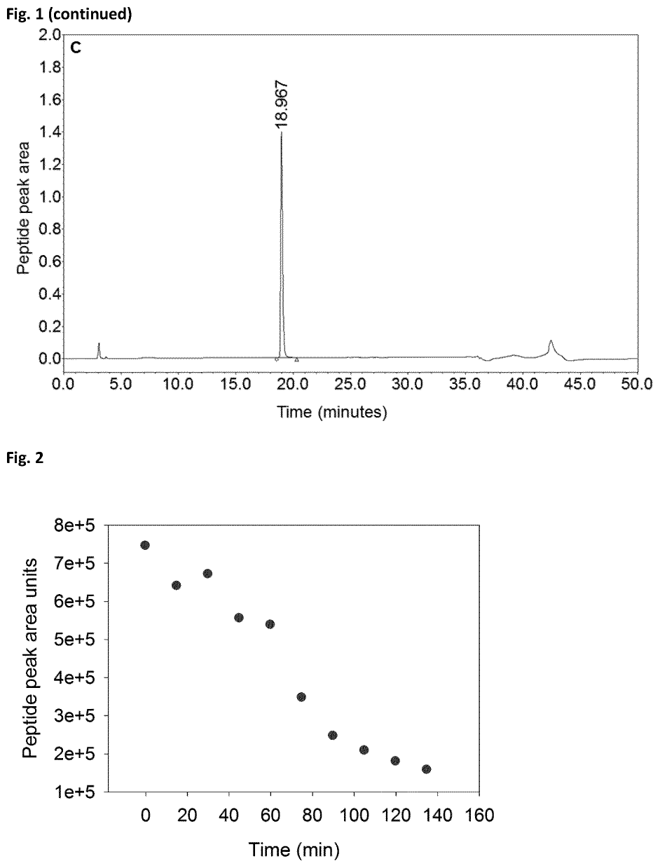

UHPLC-MS/MS method development for the peptide with amino acid sequence MFPTIPLSRLFDNAMLRAH proved challenging. Initially, the peptide peak decreased as a function of time, which we attributed to glass adsorption (see FIG. 2).

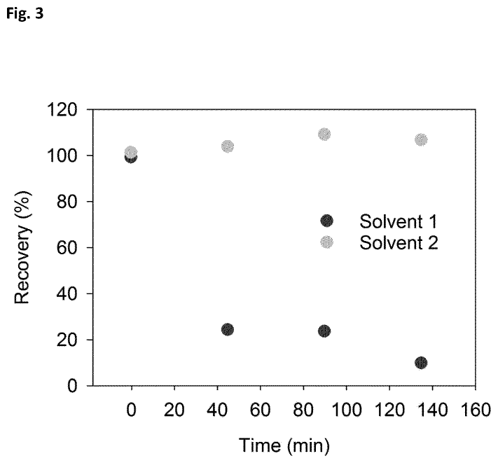

Therefore, a novel UHPLC-MS/MS compatible anti-adsorption diluent was explored. The anti-adsorption diluent contains a bovine serum albumin (BSA) hydrolysate and is obtained by boiling a BSA solution and precipitating the protein with acetonitrile, rendering the remaining solution suitable to be directly applied in UHPLC-MS/MS analysis. The previously observed glass adsorption of the peptide MFPTIPLSRLFDNAMLRAH was completely abolished by using this novel anti-adsorption diluent (see FIG. 3; anti-adsorption diluent Source 1 was used).

The traditional diluent consisting of water/acetonitrile+0.1% m/V formic acid demonstrated a decreasing peak area over time (FIG. 3) not liable to peptide degradation (see FIG. 1), hence making the quantification of this peptide unreliable.

The presence of interfering substances in the anti-adsorption diluent (Source 1) was first evaluated by injecting the anti-adsorption diluent as such. No interfering substances were observed by applying the peptide specific MRM settings. The peptide demonstrated no appreciable adsorption during more than 2 hours when kept in the presence of the anti-adsorption diluent (recovery approximately 110%), confirming the suitability of this anti-adsorption diluent for this specific peptide. Keeping this peptide in the same solvent composition but without anti-adsorption components, yielded a recovery after 2 hours below 10%.

### The Anti-Adsorption Diluent has Wide Applicability.

To further investigate the wide applicability of this novel anti-adsorption diluent, 36 peptides were selected. Selection of these peptides was made by the in-house necessity to develop bioanalytical methods for these peptides. Via a design of experiments approach, the most suitable injection solvent for each peptide was examined by UHPLC-MS/MS in the pM to nM-range. Via a D-optimal design, the quantitative influence of 4 solvent components was evaluated: (I) the fraction of water (25-100% V/V), (II) the fraction of acetonitrile (0-75% V/V), (III) the fraction of anti-adsorption diluent (Source 1), obtained as described in the materials and methods section (0-75% V/V), and (IV) the fraction of formic acid (0.025-7.5% m/V). The necessity of prior coating the vial with anti-adsorption diluent was also evaluated in the same D-optimal design model. Prior to this experiment, the LC-MS suitability of the anti-adsorption diluent was evaluated by injecting the anti-adsorption diluent as such. None of the 72 MRM's (2/peptide) showed any interference. The influence of the solvent composition on the adsorption of each peptide was evaluated using the peptide peak area as response. The model validity parameters (R² and Q²) are given together with the predicted optimum condition (vial+solvent composition) per peptide in Table 11.

Peptides were added to the (U)HPLC vial insert in 25 μL water+0.1% m/V formic acid. The other 75 μL consisted of the solvents specified in the D-optimal design. Hence, if a predicted injection solvent consists of 0.1 fraction acetonitrile and 0.9 fraction anti-adsorption diluent the predicted most suitable injection solvent of that peptide consists of 25 μL water+0.1% (m/V) formic acid+75 μL*0.1=7.5 μL acetonitrile and 75 μL*0.9=67.5 μL anti-adsorption diluent.

The model characteristic R² is a measure for the model fit whilst Q² illustrates the predictive power of the model. A model with proper predictive power is determined by a Q² value exceeding 0.5. Log(D), with D meaning the normalized distance to the target, is a quality attribute to describe the closeness to the specification limits. The absolute minimal Log(D) value is −10, meaning an on target prediction. DPMO (defects per million opportunities outside specifications) and CpK (Process Capability Indices) are probabilities of failure. The higher the DPMO or the lower the CpK, the higher the chance the predictions will be outside the specifications. The 36 individual models yield an average Q² of 0.54. All models exceed Q² of 0.1 (lowest Q²=0.162 for peptide Q52), thus all models are significant and 20 peptides out of 36 peptides have a good predictive power exceeding a Q² of 0.5.

TABLE 11
Optimal injection solvent per peptide as predicted by the Modde software. Total
injection solvent consists of 25 μL water containing the peptide + 0.1% m/V formic
acid and 75 μL solvent composed as specified by the model.

| | | | | | | Predicted most suitable injection solvent (fraction) | | | | |
|---|---|---|---|---|---|---|---|---|---|---|
| | | | | | | | | 10% | | |
| | | | | | | | | (m/V) | | Anti- |
| (Peptide | Model characteristics | | | | | | Aceto- | formic | | adsorption |
| ID | R² | Q² | Log(D) | DPMO | CpK | Vial | nitrile | acid | Water | diluent |
| Q11 | 0.922 | 0.650 | −10 | 0 | 1.687 | Uncoated | 0.1 | 0 | 0 | 0.9 |
| Q28 | 0.866 | 0.704 | −10 | 2100 | 0.902 | Coated | 0 | 0.01 | 0.58 | 0.41 |
| Q30 | 0.706 | 0.465 | −10 | 7100 | 0.796 | Uncoated | 0 | 0 | 0 | 1 |
| Q31 | 0.867 | 0.702 | −0.964 | 177700 | 0.311 | Coated | 0 | 0.16 | 0.75 | 0.10 |
| Q45 | 0.59 | 0.341 | −10 | 317600 | 0.168 | Coated | 0 | 0.44 | 0 | 0.56 |
| Q46 | 0.969 | 0.88 | −10 | 0 | 0.927 | Uncoated | 0.1 | 0 | 0 | 0.9 |
| Q47 | 0.429 | 0.359 | −0.602 | 340800 | 0.139 | Coated | 0 | 0 | 0 | 1 |
| Q53 | 0.970 | 0.893 | −10 | 0 | 0.811 | Uncoated | 0.1 | 0 | 0 | 0.9 |
| Q54 | 0.684 | 0.503 | −10 | 372300 | 0.148 | Coated | 0.17 | 0.83 | 0 | 0 |
| Q58 | 0.879 | 0.757 | −0.657 | 89300 | 0.442 | Coated | 0 | 0 | 0.46 | 0.54 |
| Q101 | 0.920 | 0.717 | −10 | 0 | 0.649 | Uncoated | 0.01 | 0 | 0 | 0.99 |
| Q102 | 0.678 | 0.464 | −10 | 6800 | 0.819 | Uncoated | 0 | 0 | 0 | 1 |
| Q171 | 0.760 | 0.610 | −1.494 | 80200 | 0.4667 | Coated | 0.16 | 0.84 | 0 | 0 |
| Q176 | 0.649 | 0.499 | −0.681 | 248600 | 0.225 | Coated | 0 | 0 | 0.39 | 0.61 |
| Q19 | 0.768 | 0.472 | −0.942 | 194900 | 0.283 | Coated | 0.78 | 0 | 0.22 | 0 |
| Q155 | 0.800 | 0.635 | −10 | 31000 | 0.604 | Coated | 1 | 0 | 0 | 0 |
| Q13 | 0.697 | 0.462 | −1.282 | 224700 | 0.254 | Uncoated | 0 | 0 | 0.53 | 0.47 |
| Q14 | 0.797 | 0.591 | −0.794 | 239500 | 0.243 | Uncoated | 0.24 | 0 | 0.76 | 0 |
| Q17 | 0.764 | 0.613 | −10 | 1900 | 0.836 | Uncoated | 0 | 0 | 1 | 0 |
| Q132 | 0.422 | 0.246 | −10 | 247900 | 0.230 | Uncoated | 0 | 0 | 1 | 0 |
| Q133 | 0.654 | 0.233 | −0.759 | 400800 | 0.114 | Uncoated | 0.37 | 0 | 0.63 | 0 |
| Q137 | 0.900 | 0.800 | −0.629 | 177600 | 0.308 | Uncoated | 0.37 | 0 | 0.63 | 0 |
| Q184 | 0.935 | 0.827 | −10 | 1600 | 0.902 | Uncoated | 0.34 | 0.64 | 0.02 | 0 |
| Q210 | 0.804 | 0.579 | −10 | 64800 | 0.505 | Uncoated | 0 | 0 | 1 | 0 |
| Q49 | 0.669 | 0.215 | −10 | 500 | 0.806 | Uncoated | 0 | 0 | 0 | 1 |
| Q125 | 0.557 | 0.308 | −10 | 191300 | 0.287 | Uncoated | 0 | 0 | 0 | 1 |
| Q138 | 0.858 | 0.651 | −10 | 2500 | 0.918 | Coated | 0 | 0.27 | 0.38 | 0.35 |
| Q208 | 0.933 | 0.864 | −10 | 260800 | 0.228 | Coated | 0 | 0.59 | 0.41 | 0 |
| Q206 | 0.778 | 0.615 | −0.611 | 264500 | 0.205 | Coated | 0 | 0 | 0.95 | 0.05 |
| Q34 | 0.839 | 0.701 | −10 | 0 | 0.933 | Coated | 1 | 0 | 0 | 0 |
| Q50 | 0.668 | 0.415 | −0.602 | 369000 | 0.112 | Uncoated | 1 | 0 | 0 | 0 |
| Q52 | 0.565 | 0.162 | −10 | 41800 | 0.131 | Uncoated | 0 | 0 | 1 | 0 |
| Q160 | 0.702 | 0.466 | −10 | 42000 | 0.564 | Uncoated | 0 | 0 | 0 | 1 |
| Q162 | 0.498 | 0.220 | −10 | 102400 | 0.419 | Coated | 1 | 0 | 0 | 0 |
| Q192 | 0.471 | 0.296 | −10 | 362900 | 0.118 | Coated | 1 | 0 | 0 | 0 |
| Q193 | 0.738 | 0.504 | −10 | 0 | 1.483 | Uncoated | 0 | 0 | 0 | 1 |

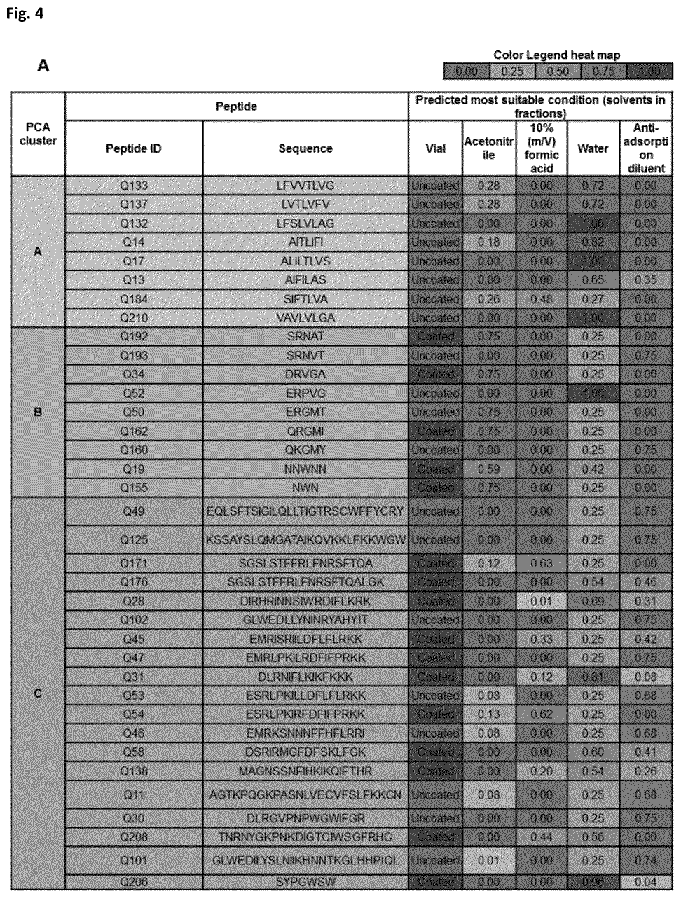

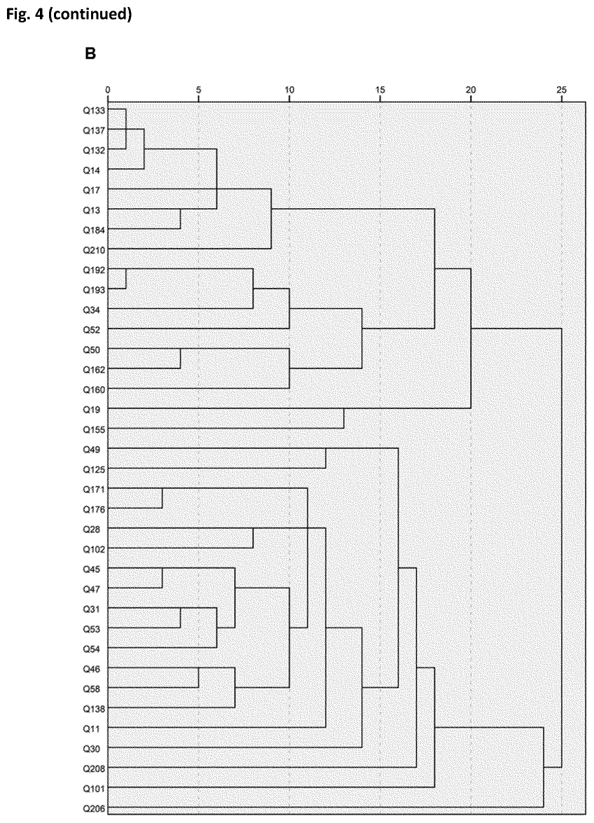

Out of 36 peptides, modeling predicted the anti-adsorption diluent Source 1 to be beneficial to 19 peptides (approximately 50%). Coating of the vials with anti-adsorption diluent Source 1 also proved beneficial for 16 out of 36 peptides, with or without simultaneous presence of the anti-adsorption diluent Source 1 in the injection solvent (see FIG. 4). In total, 75% of the evaluated peptides benefited from the presence of the anti-adsorption diluent Source 1 (27 out of 36). For example, peptide EQLSFTSIGILQLLTIGTRSCWFFYCRY (Q49; SEQ ID No. 26) has a predicted relative optimal injection solvent consisting of 75% anti-adsorption diluent (see FIG. 4-5).

### Anti-Adsorption Diluent Applicability.

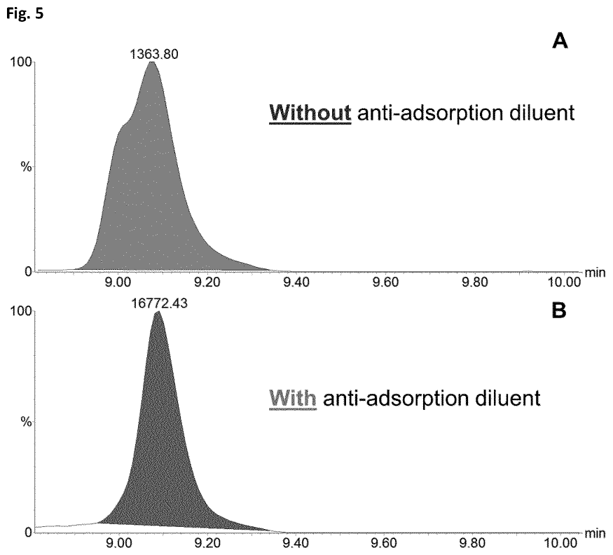

Principal component analysis of the 36 peptides was carried out with 446 descriptors for each peptide. The score plot is depicted in FIG. 6 and shows 3 clusters for PC1-PC2. These 3 “physicochemical” clusters are then visualized in the HCA heat map in FIG. 4. While cluster A peptides generally do not benefit from the anti-adsorption diluent (i.e. only 1 of the 8 peptides in cluster A benefitted), all cluster C peptides (i.e. 19 peptides) do need anti-adsorption diluent to minimize adsorption to the glass container (either in the injection solvent or by coating the vial). Cluster B peptides generally required anti-adsorption diluent (i.e. 7 out of 9 peptides), with only 2 structurally related peptides not requiring anti-adsorption diluent (i.e. Q50 and Q52).

By plotting PC1 versus PC3, 2 “physicochemical” clusters are distinguished. These 2 clusters concur with the beneficial effect of the anti-adsorption diluent. In cluster E, all peptides (except Q206, i.e. 18 out of 19 peptides) do benefit from the anti-adsorption diluent to reduce glass adsorption. On the other hand, the peptides being part of cluster D do generally not benefit from this anti-adsorption diluent. The first 3 Principal Components of the model account for goodness of fit of approximately 60% and a predictive power of approximately 50%, rendering it an appropriate model.

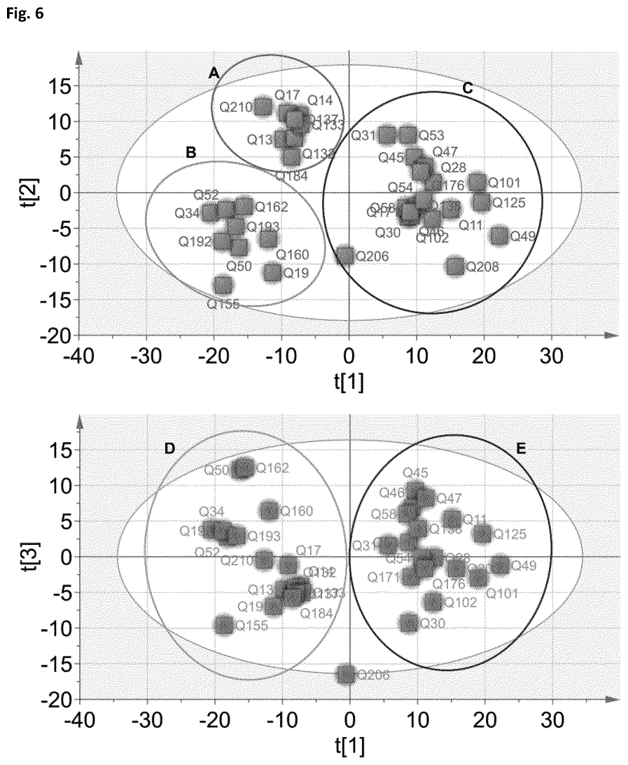

The loading plot (data not shown) shows that both the first and second principal components are majorly determined by 3D MoRSE, 2D atom pairs, 2D autocorrelation, GETAWAY, Burden eigen values and geometrical descriptors. These descriptors do divide the peptides into 2 clusters when PC1 is plotted against PC3. Amongst these descriptors, log P descriptors are found to determine the first principal component amongst others. Examining the Log P of the investigated peptides showed that on average the peptides in cluster C and B of FIG. 6 are hydrophilic (i.e. a negative calculated octanol/water partition coefficient), while those in cluster A are hydrophobic (i.e. a positive calculated octanol/water partition coefficient). The peptides in cluster B and C differ amongst each other in the peptide length and the best chromatographic separation system. The peptides in cluster B are on average 5 amino acids long and are chromatographed using the HILIC amide column system whilst these in cluster C are all exceeding 10 amino acids in length and are compatible with a C₁₈ chromatographic system for analysis. Cluster D and E (see FIG. 6 lower panel) distinguish peptides that generally benefit from the anti-adsorption diluent versus those who do mostly not. Cluster D in PC1-PC3 is composed of the peptides from cluster A and B in the PC1-PC2 (see FIG. 6 upper panel). These peptides positioned in cluster A are characterized by a median molecular weight of 750 Da, an isoelectric point of 5.9, the absence of amino acids with chargeable side chains and are predominantly composed of hydrophobic amino acids (see Table 12 and 13 and FIG. 7).

TABLE 12
Selected peptide descriptors.

| | | Descriptors | | | | |
|---|---|---|---|---|---|---|
| Peptide | | | | | hydrogen | hydrogen |
| Peptide | | Moleculair | | | donor | acceptor |
| ID | Sequence | weight | pl | logP | bounds | bounds |
| Q133 | LFVVTLVG | 847.2 | 5.99 | 1.47 | 11 | 18 |
| Q137 | LVTLVFV | 790.14 | 5.98 | 2.55 | 10 | 16 |
| Q132 | LFSLVLAG | 819.14 | 5.93 | 0.51 | 11 | 18 |
| Q14 | AITLIFI | 790.14 | 5.98 | 2.54 | 10 | 16 |
| Q17 | ALILTLVS | 829.19 | 5.92 | 0.26 | 12 | 19 |
| Q13 | AIFILAS | 734.00 | 5.85 | 0.86 | 10 | 16 |
| Q184 | SIFTLVA | 750.02 | 5.71 | 0.09 | 11 | 17 |
| Q210 | VAVLVLGA | 741.07 | 5.94 | 0.16 | 10 | 17 |
| Q192 | SRNAT | 547.06 | 9.35 | 5.73 | 15 | 18 |
| Q193 | SRNVT | 575.72 | 9.37 | 4.86 | 15 | 18 |
| Q34 | DRVGA | 516.64 | 6.07 | -3.37 | 12 | 16 |
| Q52 | ERPVG | 556.71 | 6.13 | -2.89 | 11 | 16 |
| Q50 | ERGMT | 592.77 | 6.18 | -4.38 | 13 | 17 |
| Q162 | QRGMI | 603.85 | 9.68 | -3.60 | 13 | 16 |
| Q160 | QKGMY | 625.84 | 8.62 | -3.30 | 12 | 15 |
| Q19 | NNWNN | 660.73 | 5.29 | -7.17 | 16 | 19 |
| Q155 | NWN | 432.39 | 5.43 | -3.12 | 10 | 11 |
| Q49 | EQLSFTSIGILQLLTIGTRSCW | 3347.39 | 8.45 | -5.76 | 52 | 77 |
| | FFYCRY | | | | | |
| Q125 | KSSAYSLQMGATAIKQVKKLF | 2986.04 | 10.65 | -11.16 | 51 | 68 |
| | KKWGW | | | | | |
| Q171 | SGSLSTFFRLFNRSFTQA | 2066.59 | 10.81 | -8.50 | 38 | 53 |
| Q176 | SGSLSTFFRLFNRSFTQALGK | 2365.03 | 11.00 | -9.69 | 43 | 60 |
| Q28 | DIRHRINNSIWRDIFLKRK | 2481.30 | 11.27 | -8.28 | 50 | 63 |
| Q102 | GLWEDLLYNINRYAHYIT | 2254.83 | 5.44 | -3.58 | 36 | 53 |
| Q45 | EMRISRIILDFLFLRKK | 2179.07 | 10.78 | -1.84 | 38 | 51 |
| Q47 | EMRLPKILRDFIFPRKK | 2188.08 | 10.79 | -3.29 | 37 | 51 |
| Q31 | DLRNIFLKIKFKKK | 1791.55 | 10.82 | -2.91 | 33 | 41 |
| Q53 | ESRLPKILLDFLFLRKK | 2116.96 | 10.56 | -1.58 | 35 | 49 |
| Q54 | ESRLPKIRFDFIFPRKK | 2177.96 | 10.84 | -3.58 | 38 | 52 |
| Q46 | EMRKSNNNFFHFLRRI | 2109.76 | 11.01 | -8.55 | 41 | 53 |
| Q58 | DSRIRMGFDFSKLFGK | 1904.5 | 10.17 | -6.26 | 34 | 47 |
| Q138 | MAGNSSNFIHKIKQIFTHR | 2229.93 | 10.73 | -11.44 | 40 | 55 |
| Q11 | AGTKPQGKPASNLVECVFSLF | 2667.54 | 9.94 | -14.42 | 43 | 66 |
| | KKCN | | | | | |
| Q30 | DLRGVPNPWGWIFGR | 1770.27 | 9.69 | -4.19 | 28 | 41 |
| Q208 | TNRNYGKPNKDIGTCIWSGFR | 2668.4 | 9.87 | -15.37 | 49 | 68 |
| | HC | | | | | |
| Q101 | GLWEDILYSLNIIKHNNTKGLH | 3168.13 | 6.92 | -11.01 | 49 | 75 |
| | HPIQL | | | | | |
| Q206 | SYPGWSW | 882.04 | 5.69 | -1.33 | 13 | 18 |

TABLE 13
Peptide amino acid composition per amino acid class.

| | | Amino acid side chain composition | | | | | | | | | |
|---|---|---|---|---|---|---|---|---|---|---|---|
| | | (absolute number) | | | | | Amino acid side chain composition (%) | | | | |
| | | | | | hydro- | | | | | hydro- | |
| | | | | | phobic | | | | | phobic | |
| | | | | | amino | | | | | amino | |
| | | posi- | | | acids | | posi- | | | acids | |
| | | tive | | polar | (A, I, | Miscel- | tive | | polar | (A, I, | Miscel- |
| Peptide | | charge | negative | uncharged | L, M, | laneous | charge | negative | uncharged | L, M, | laneous |
| Peptide | | (R, H or | charge | (S, T, | F, W, | (C, G | (R, H | charge | (S, T, | F, W, | (C, G |
| ID | Sequence | K) | (D or E) | N or Q) | Y or V) | or P) | or K)) | (D or E) | N or Q) | Y or V) | or P) |
| Q133 | LFVVTLVG | 0 | 0 | 1 | 6 | 1 | 0.0 | 0.0 | 12.5 | 75.0 | 12.5 |
| Q137 | LVTLVFV | 0 | 0 | 1 | 6 | 0 | 0.0 | 0.0 | 14.3 | 85.7 | 0.0 |
| Q132 | LFSLVLAG | 0 | 0 | 1 | 6 | 1 | 0.0 | 0.0 | 12.5 | 75.0 | 12.5 |
| Q14 | AITLIFI | 0 | 0 | 1 | 6 | 0 | 0.0 | 0.0 | 14.3 | 85.7 | 0.0 |
| Q17 | ALILTLVS | 0 | 0 | 2 | 6 | 0 | 0.0 | 0.0 | 25.0 | 75.0 | 0.0 |
| Q13 | AIFILAS | 0 | 0 | 1 | 6 | 0 | 0.0 | 0.0 | 14.3 | 85.7 | 0.0 |
| Q184 | SIFTLVA | 0 | 0 | 2 | 5 | 0 | 0.0 | 0.0 | 28.6 | 71.4 | 0.0 |
| Q210 | VAVLVLGA | 0 | 0 | 0 | 7 | 1 | 0.0 | 0.0 | 0.0 | 87.5 | 12.5 |
| Q192 | SRNAT | 1 | 0 | 3 | 1 | 0 | 20.0 | 0.0 | 60.0 | 20.0 | 0.0 |
| Q193 | SRNVT | 1 | 0 | 3 | 1 | 0 | 20.0 | 0.0 | 60.0 | 20.0 | 0.0 |
| Q34 | DRVGA | 1 | 1 | 0 | 2 | 1 | 20.0 | 20.0 | 0.0 | 40.0 | 20.0 |
| Q52 | ERPVG | 1 | 1 | 0 | 1 | 2 | 20.0 | 20.0 | 0.0 | 20.0 | 40.0 |
| Q50 | ERGMT | 1 | 1 | 1 | 1 | 1 | 20.0 | 20.0 | 20.0 | 20.0 | 20.0 |
| Q162 | QRGMI | 1 | 0 | 1 | 2 | 1 | 20.0 | 0.0 | 20.0 | 40.0 | 20.0 |
| Q160 | QKGMY | 1 | 0 | 1 | 2 | 1 | 20.0 | 0.0 | 20.0 | 40.0 | 20.0 |
| Q19 | NNWNN | 0 | 0 | 4 | 1 | 0 | 0.0 | 0.0 | 80.0 | 20.0 | 0.0 |
| Q155 | NWN | 0 | 0 | 2 | 1 | 0 | 0.0 | 0.0 | 66.7 | 33.3 | 0.0 |
| Q49 | EQLSFTSIGIL | 2 | 1 | 8 | 13 | 4 | 7.1 | 3.6 | 28.6 | 46.4 | 14.3 |
| | QLLTIGTRSCW | | | | | | | | | | |
| | FFYCRY | | | | | | | | | | |
| Q125 | KSSAYSLQMGA | 6 | 0 | 6 | 12 | 2 | 23.1 | 0.0 | 23.1 | 46.2 | 7.7 |
| | TAIKQVKKLFK | | | | | | | | | | |
| | KWGW | | | | | | | | | | |
| Q171 | SGSLSTFFRLF | 2 | 0 | 8 | 7 | 1 | 11.1 | 0.0 | 44.4 | 38.9 | 5.6 |
| | NRSFTQA | | | | | | | | | | |
| Q176 | SGSLSTFFRLF | 3 | 0 | 8 | 8 | 2 | 14.3 | 0.0 | 38.1 | 38.1 | 9.5 |
| | NRSFTQALGK | | | | | | | | | | |
| Q28 | DIRHRINNSIW | 7 | 2 | 3 | 7 | 0 | 36.8 | 10.5 | 15.8 | 36.8 | 0.0 |
| | RDIFLKRK | | | | | | | | | | |
| Q102 | GLWEDLLYNIN | 2 | 2 | 3 | 10 | 1 | 11.1 | 11.1 | 16.7 | 55.6 | 5.6 |
| | RYAHYIT | | | | | | | | | | |
| Q45 | EMRISRIILDF | 5 | 2 | 1 | 9 | 0 | 29.4 | 11.8 | 5.9 | 52.9 | 0.0 |
| | LFLRKK | | | | | | | | | | |
| Q47 | EMRLPKILRDF | 6 | 2 | 0 | 7 | 2 | 35.3 | 11.8 | 0.0 | 41.2 | 11.8 |
| | IFPRKK | | | | | | | | | | |
| Q31 | DLRNIFLKIKF | 6 | 1 | 1 | 6 | 0 | 42.9 | 7.1 | 7.1 | 42.9 | 0.0 |
| | KKK | | | | | | | | | | |
| Q53 | ESRLPKILLDF | 5 | 2 | 1 | 8 | 1 | 29.4 | 11.8 | 5.9 | 47.1 | 5.9 |
| | LFLRKK | | | | | | | | | | |
| Q54 | ESRLPKIRFDF | 6 | 2 | 1 | 6 | 2 | 35.3 | 11.8 | 5.9 | 35.3 | 11.8 |
| | IFPRKK | | | | | | | | | | |
| Q46 | EMRKSNNNFFH | 5 | 1 | 4 | 6 | 0 | 31.3 | 6.3 | 25.0 | 37.5 | 0.0 |
| | FLRRI | | | | | | | | | | |
| Q58 | DSRIRMGFDFS | 4 | 2 | 2 | 6 | 2 | 25.0 | 12.5 | 12.5 | 37.5 | 12.5 |
| | KLFGK | | | | | | | | | | |
| Q138 | MAGNSSNFIHK | 5 | 0 | 6 | 7 | 1 | 26.3 | 0.0 | 31.6 | 36.8 | 5.3 |
| | IKQIFTHR | | | | | | | | | | |
| Q11 | AGTKPQGKPAS | 4 | 1 | 6 | 8 | 6 | 16.0 | 4.0 | 24.0 | 32.0 | 24.0 |
| | NLVECVFSLFK | | | | | | | | | | |
| | KCN | | | | | | | | | | |
| Q30 | DLRGVPNPWGW | 2 | 1 | 1 | 6 | 5 | 13.3 | 6.7 | 6.7 | 40.0 | 33.3 |
| | IFGR | | | | | | | | | | |
| Q208 | TNRNYGKPNKD | 5 | 1 | 6 | 5 | 6 | 21.7 | 4.3 | 26.1 | 21.7 | 26.1 |
| | IGTCIWSGFRH | | | | | | | | | | |
| | C | | | | | | | | | | |
| Q101 | GLWEDILYSLN | 5 | 2 | 6 | 11 | 3 | 18.5 | 7.4 | 22.2 | 40.7 | 11.1 |
| | IIKHNNTKGLH | | | | | | | | | | |
| | HPIQL | | | | | | | | | | |
| Q206 | SYPGWSW | 0 | 0 | 2 | 3 | 2 | 0.0 | 0.0 | 28.6 | 42.9 | 28.6 |

Peptides being part of cluster B are characterized by a median molecular weight of 576 Da, have an average isoelectric point of 6.2, and most peptides possess at least 1 chargeable amino acid side chain (positive or negative charge). Peptides in cluster E on the other hand are considerably larger than these in cluster D with a median molecular mass of 2188 Da, do also have a higher isoelectric point (median 10.7) and multiple chargeable amino acid side chains (positive or negative charge). The percentage of polar uncharged amino acids does not differ amongst the different clusters.

Various mechanisms have been attributed to glass adsorption, such as hydrogen bound formation with the glass silanol functional moieties. Therefore, the number of hydrogen bound donors (bound to oxygen or nitrogen atoms) and acceptors (oxygen or nitrogen atoms) was evaluated. The peptides in cluster E have a median number of hydrogen bound donors of 38 and hydrogen bound acceptors of 53 respectively. Peptides from cluster D on the other hand, have a medium number of hydrogen bound donors of 11 and acceptors of respectively 17 without considerable difference among the peptides being part of cluster A and B regarding hydrogen bound donors or acceptors. Given the physicochemical nature of glass adsorption, it is hypothesized that peptides that own a relative high number of hydrogen donors and/or acceptors do generally benefit from the anti-adsorption diluent. The peptide MFPTIPLSRLFDNAMLRAH, that also benefits from the anti-adsorption diluent (see proof of principle experiments), has a calculated number of hydrogen bound donors of 32 and hydrogen bound acceptors of 50, thus in line with the observation made for the proof of concept peptides. In general, peptides having a relative high isoelectric point, molecular mass and/or relative large number of hydrogen donor and acceptor bounds might be considered as candidate peptides benefiting this anti-adsorption diluent to reduce glass adsorption.

### The Anti-Adsorption Diluent is a Peptide Mixture

The identity of the constituting peptides in anti-adsorption diluent Source 1 was elucidated via in silico de novo peptide sequencing, following high resolution mass spectroscopy. In total, 398 peptides were identified. The smallest peptide is composed of 4 amino acids and the largest of 22 amino acids. The 10 most abundant peptides are illustrated in Table 14. The peptide mixture coming from the specific protein treatment has remarkable anti-adsorption properties.

The matrix effect of the anti-adsorption diluents (i.e. Source 1, Source 2, and Source 3) was examined with 1 exemplary peptide (EMRKSNNNFFHFLRRI; Q46) and evaluated by post-column infusion of the aforementioned peptide. More specific, the peptide Q46 was post-column infused at 5 μL/min and a concentration of 1 μM. Concomitantly whilst the peptide was infused, the anti-adsorption diluents were chromatographically separated (10 μL injection volume) using the peptide specific chromatographic and mass spectrometric settings.

The matrix effects on peptide Q46 were examined using the UHPLC-MS/MS settings outlined in the ‘Proof of concept (pivotal experiment)’ section. Each anti-adsorption diluent (3 sources) was injected in triplicate for each peptide and matrix effects were monitored using the peptide quantifier MRM as previously provided.

TABLE 14
Amino acid composition of 10 most abundant peptides in the anti-adsorption diluent.
Amino acid sequence
THEMETHVADLS (SEQ ID No. 38)
HTEMETHVADLS (SEQ ID No. 39)
THEMETHAVDLS (SEQ ID No. 40)
WVNDNEEGFFSLN (SEQ ID No. 41)
WVNDNEEGFFSAR (SEQ ID No. 42)
ADVNDNEEGFFSAR (SEQ ID No. 43)
ERNDNEEGFFSAR (SEQ ID No. 44)
AVDNDNEEGFFSAR (SEQ ID No. 45)
QRDDNEEGFFSAR (SEQ ID No. 46)
QAEADNEEGFFSAR (SEQ ID No. 47)

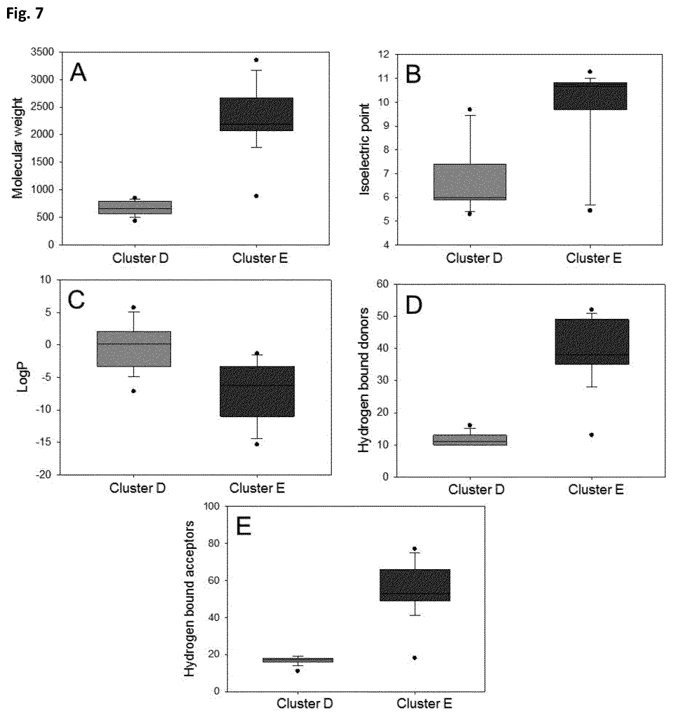

Matrix effects were observed (as demonstrated in FIG. 8) but the peptide elutes in an area unaffected by matrix effects caused by the anti-adsorption diluent.

Bovine Serum Albumin Origin does not Significantly Influence Functionality

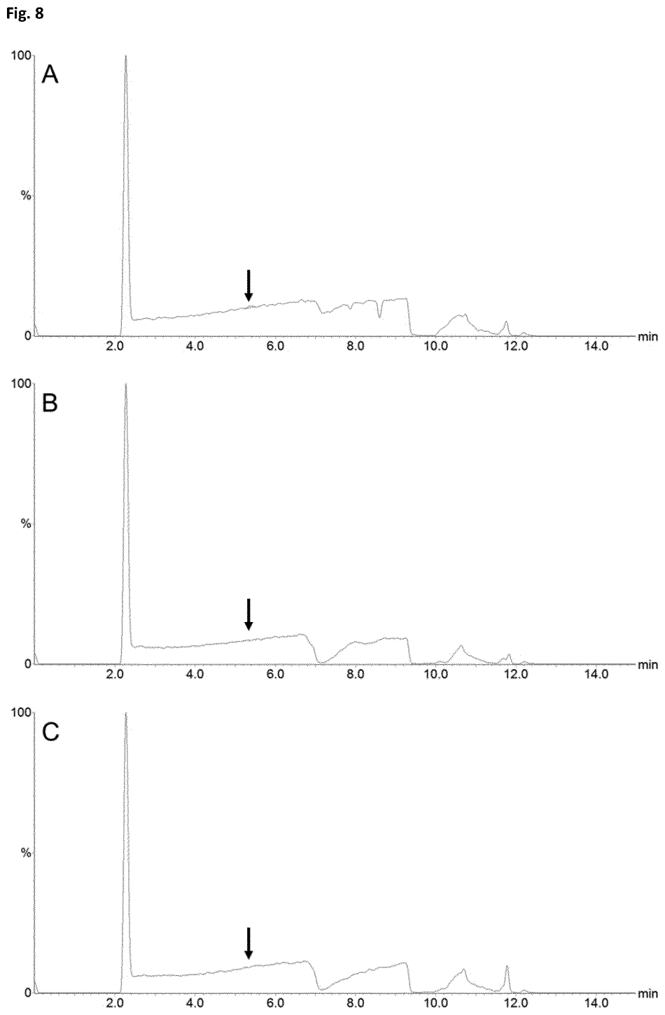

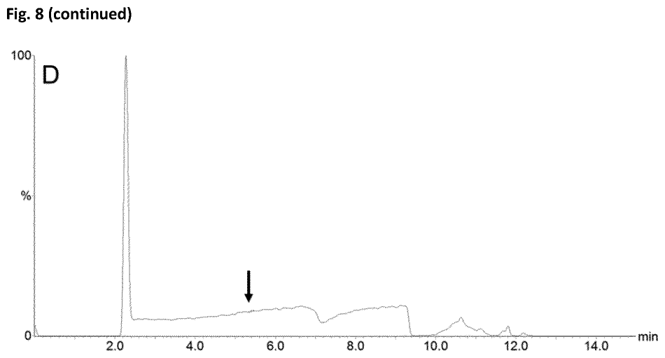

The pilot and pivotal experiments were carried out using anti-adsorption diluent originating from Source 1. Since Bovine Serum Albumin can differ amongst suppliers, anti-adsorption diluent was prepared originating from 3 sources, referred to as Source 1, Source 2, and Source 3. To evaluate the effect of the Bovine Serum Albumin origin on the anti-adsorption functionality, (I) the effect of the anti-adsorption diluent origin on adsorption of the peptide MFPTIPLSRLFDNAMLRAH (100 ng/mL analytical concentration) was evaluated and (II) the peptide solution was repeatedly transferred from one insert to another (5 inserts in total) with subsequent analysis of the 5^{th} insert. FIG. 9 demonstrates that, regardless of the Bovine Serum Albumin source, all anti-adsorption diluents do outperform the condition without anti-adsorption diluent (i.e. the solvent condition). Additionally, the peptide peak area after repeatedly transferring the analytical solution to 5 inserts in total, varies from approximately 80 to 95% of the peptide peak area without transfer. On the other hand, when the peptide is transferred 5 times from one insert to another in 50/50 V/V water/acetonitrile+0.1% m/V formic acid, the peptide peak area is approximately 10% of the peptide peak area without transferring the analytical solution from one insert to the others. Representative chromatograms are provided in FIG. 10.

### Anti-Adsorption Diluent Applicability for Polypropylene Inserts

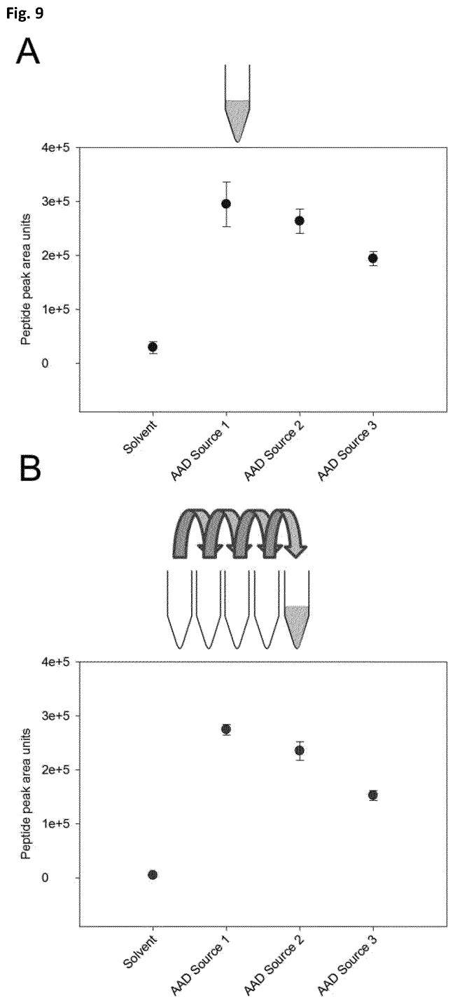

Previous experiments were all conducted using glass inserts. Plastic vials and inserts are also widely used for the chromatography of peptides. Therefore, the applicability of the anti-adsorption diluent was evaluated using a polypropylene insert. The peptide MFPTIPLSRLFDNAMLRAH (100 ng/mL) was either analyzed using a glass or polypropylene insert with or without anti-adsorption diluent (see FIG. 11).

As can be observed from FIG. 11, the anti-adsorption diluent did improve the peptide peak area in either glass or polypropylene inserts. The effect of the anti-adsorption diluent was most pronounced when glass inserts were used. Nevertheless, when polypropylene was used together with anti-adsorption diluent, the peptide peak area still did increase by approximately 30% compared to the polypropylene insert without anti-adsorption diluent, thus demonstrating the utility of the anti-adsorption diluent. Evidently, this is peptide-specific and thus should be evaluated for every peptide until for example a satisfactory detection limit is obtained or a vial+/−anti-adsorption combination is found that is as little as possible affected by adsorption.

### AAD as a Function of Process Variables

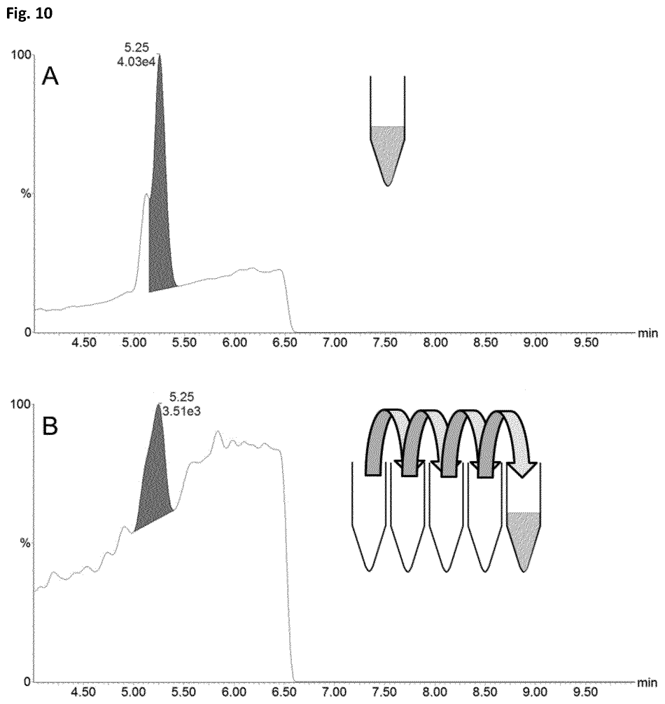

The main variables that contribute to the AAD are: (I) protein source, (II) organic solvent, (III) acid, and (IV) heating. When changing acetonitrile to either isopropanol or denatured ethanol, a comparable functionality is observed as does simultaneous alteration of the protein source (see FIG. 12).

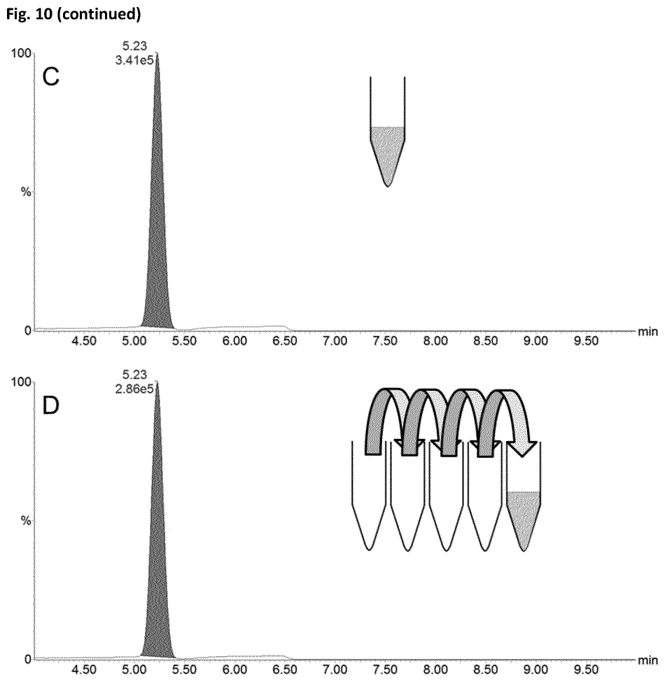

Changing the acid does also influence (but not abolish) the functionality as demonstrated in FIG. 13. Some acids such as trifluoroacetic acid do even outperform the anti-adsorption solution prepared using formic acid.

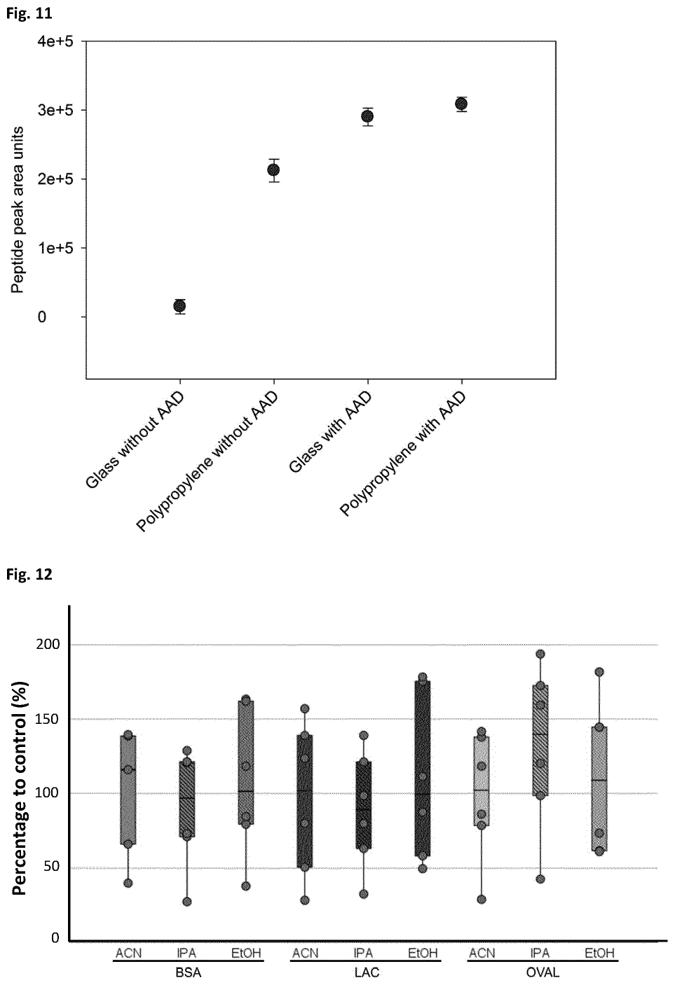

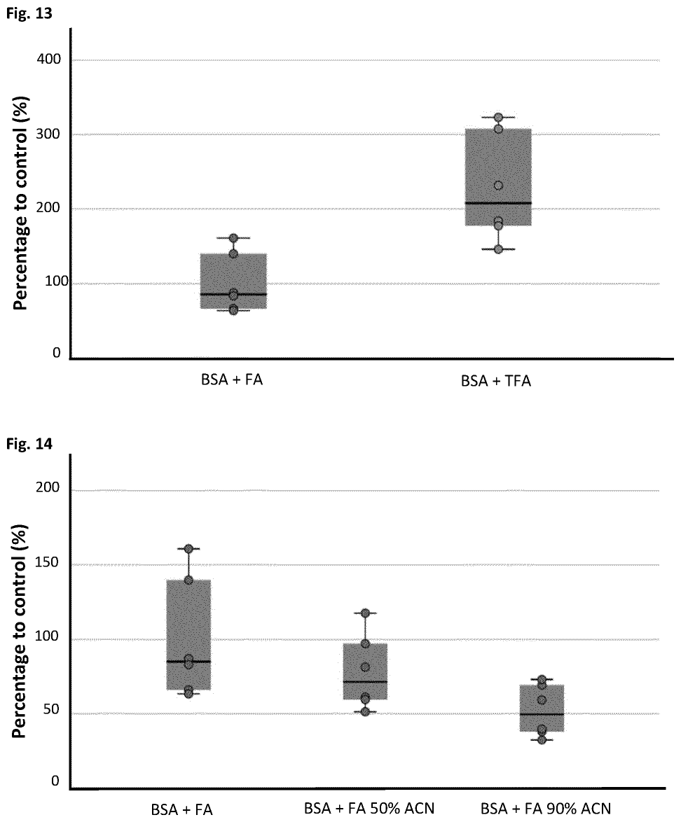

Altering the organic solvent percentage does also render functional AAD (see FIG. 14). The presence of acid augment the anti-adsorption activity, but is not a strict dichotomic requirement. Thus, the presence of an acid is not mandatory in a binary classification-based decision. As can be appreciated from FIG. 15, preparation of the anti-adsorption solution without an acid, rendered a comparable functionality as demonstrated for peptide Q46.

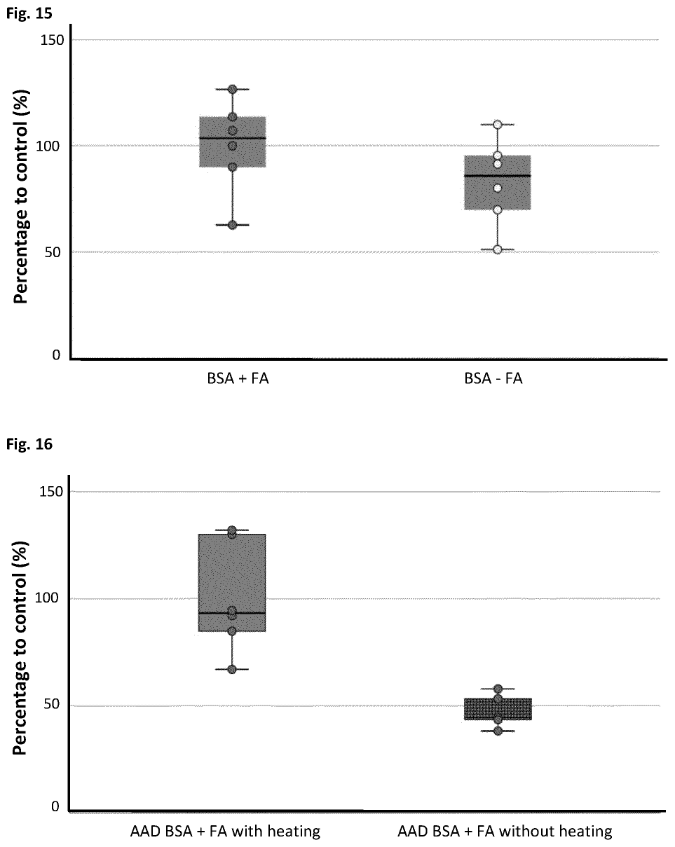

The variable heating was investigated by preparing AAD with and without heating while all other parameters were kept unchanged. As can be appreciated from FIG. 16, without heating a still functional AAD was obtained, where heating does augment the functionality.

### CONCLUSIONS

Peptide analysis in the low concentration range can be challenging due to adsorption to the analytical vials. Various anti-adsorption approaches are already reported. Here, a new approach is added to the set of anti-adsorption methods. This anti-adsorption diluent is based on a protein (e.g. bovine serum albumin) hydrolysate and is UHPLC-MS/MS compatible. Additionally, the diluent is easily formic acid) to increase its use-flexibility. The suitability to alleviate the adsorption of peptides to glass vials was demonstrated in a set of 36 representative peptides.

This anti-adsorption diluent could also have a potential place in the peptidomics field. Since some peptides are abundant in a low concentration and might show adsorption to the glass vial, they might be missed during untargeted peptide analysis. By applying this anti-adsorption diluent, this analytical artifact can be decreased. In targeted mode, the anti-adsorption diluent did not interfere in the peptide specific MRM settings of the 36 investigated peptides. As such, this anti-adsorption diluent has a place in different peptide analysis applications, such as targeted or untargeted peptidomics. Altering the process variables, i.e. (I) protein, (II) organic solvent and solvent percentage, (III) kind of acid and acid presence, and (IV) heating does in all cases render a functional AAD. However, eliminating the heating step as does altering the organic solvent percentage renders a less, but still, functional AAD.

### REFERENCES

[1] Craik, D.; Fairlie, D.; Liras, S.; Price, D. Chem. Biol. Drug Des. The future of peptide-based drugs. 2013, 81, 136-147.
[2] Pezeshki, A.; Vergote, V.; Van Dorpe, S.; Baert, B.; Burvenich, C.; Popkov, A.; De Spiegeleer, B. Adsorption of peptides at the sample drying step: Influence of solvent evaporation technique, vial material and solution additive. J. Pharm. Biomed. Anal. 2009, 49(3), 607-612.
[3] Kristensen, K.; Henriksen, J. R.; Andresen, T. L. Adsorption of cationic peptides to solid surfaces of glass and plastic. PLoS One. 2015, 10(5), e0122419.
[4] Stejskal, K.; Potesil, D.; Zdrahal, Z. Suppression of peptide sample losses in autosampler vials. J. Proteome Res. 2013, 12(6), 3057-3062.
[5] Maes, K.; Smolders, I.; Michotte, Y.; Van Eeckhaut, A. Strategies to reduce aspecific adsorption of peptides and proteins in liquid chromatography-mass spectrometry based bioanalyses: an overview. J. Chromatogr. A. 2014, 1358, 1-13.
[6] Fouda, H.; Nocerini, M.; Schneider, R.; Gedutis, C. Quantitative analysis by high-performance liquid chromatography atmospheric pressure chemical ionization mass spectrometry: The determination of the renin inhibitor CP-80,794 in human serum. J. Am. Soc. Mass Spectrom. 1991, 2, 164-167.
[7] Chambers, E. E.; Legido-Quigley, C.; Smith, N.; Fountain, K. J. Development of a fast method for direct analysis of intact synthetic insulins in human plasma: the large peptide challenge. Bioanalysis. 2013, 5, 65-81.
[8] van den Broek, I.; Niessen, W. M.; van Dongen, W. D. Bioanalytical LC-MS/MS of protein-based biopharmaceuticals. J. Chromatogr. B. 2013, 929, 161-179.
[9] Goebel-Stengel, M.; Stengel, A.; Tache, Y.; Reeve, J. R. Jr. The importance of using the optimal plasticware and glassware in studies involving peptides. Anal. Biochem. 2011, 414, 38-46.
[10] Reeve, J. R.; Wu, S. V.; Keire, D. A.; Faull, K.; Chew, P.; Solomon, T. E.; Green, G. M.; Coskun, T. Differential bile-pancreatic secretory effects of CCK-58 and CCK-8. Am. J. Physiol. Gastrointest. Liver Physiol. 2004, 286, G395-G402.
[11] Persson, D.; Thorén, P. E.; Herner, M.; Lincoln, P.; Norden, B. Application of a novel analysis to measure the binding of the membrane-translocating peptide penetratin to negatively charged liposomes. Biochemistry. 2003, 42, 421-429.
[12] Prakash, A.; Rezai, T.; Krastins, B.; Sarracino, D.; Athanas, M.; Russo, P.; Ross, M. M.; Zhang, H.; Tian, Y.; Kulasingam, V.; Drabovich, A. P.; Smith, C.; Batruch, I.; Liotta, L.; Petricoin, E.; Diamandis, E. P.; Chan, D. W.; Lopez, M. F. Platform for establishing interlaboratory reproducibility of selected reaction monitoring-based mass spectrometry peptide assays. J. Proteome Res. 2010, 9, 6678-6688.
[13] Prakash, A. Rezai, T.; Krastins, B.; Sarracino, D.; Athanas, M.; Russo, P.; Zhang, H.; Tian, Y.; Li, Y.; Kulasingam, V.; Drabovich, A.; Smith, C. R.; Batruch, I.; Oran, P. E.; Fredolini, C.; Luchini, A.; Liotta, L.; Petricoin, E.; Diamandis, E. P.; Chan, D. W.; Nelson, R.; Lopez, M. F. Interlaboratory reproducibility of selective reaction monitoring assays using multiple upfront analyte enrichment strategies. J. Proteome Res. 2012, 11, 3986-3995.
[14] van Platerink, C. J.; Janssen, H. G.; Horsten, R.; Haverkamp, J. Quantification of ACE inhibiting peptides in human plasma using high performance liquid chromatography-mass spectrometry. J. Chromatogr. B. 2006, 830, 151-157.
[15] Kippen, A. D.; Cerini, F.; Vadas, L.; Stöcklin, R.; Vu, L.; Offord, R. E.; Rose, K. Development of an Isotope Dilution Assay for Precise Determination of Insulin, C-peptide, and Proinsulin Levels in Non-diabetic and Type II Diabetic Individuals with Comparison to Immunoassay. J. Biol. Chem. 1997, 272, 12513-12522.
[16] Suelter, C. H.; DeLuca, M. How to prevent losses of protein by glass adsorption to glass and plastic. Anal. Biochem. 1983, 135, 112-119.
[17] Kramer, K. J.; Dunn, P. E.; Peterson, R. C.; Seballos, H. L.; Sanburg, L. L.; Law, J. H. Purification and characterization of the carrier protein for juvenile hormone from the hemolymph of the tobacco hornworn Manduca sexta Johannson (Lepidoptera: Sphingidae). J. Biol. Chem. 1976, 251, 4979-4985.
[18] Wynendaele, E.; Gevaert, B.; Stalmans, S.; Verbeke, F.; De Spiegeleer, B. Biopolymers. Exploring the chemical space of quorum sensing peptides. 2015, 104, 544-551.
[19] Bin, M.; Kaizhong, Z.; Christopher, H.; Chengzhi, L.; Ming, L.; Amanda, D. K.; Gilles L. PEAKS: Powerful Software for Peptide De Novo Sequencing by MS/MS. Rapid Communications in Mass Spectrometry, 17(20):2337-2342. 2003.

1-18. (canceled)

19. A process for preparing an anti-adsorption solution, the process comprising:
(i) preparing a solution comprising one or more proteins and/or peptides at a concentration from 25 ng/mL to 1 g/mL in water;
(ii) diluting the solution of (i) to a concentration of 2.5 ng/mL to 0.1 g/mL of the one or more proteins and/or peptides by adding to the solution a 0.1% to 99% (v/v) organic solvent solution to obtain a diluted solution;
(iii) cooling the diluted solution obtained in (ii) to obtain a cooled solution;
(iv) separating and isolating the cooled solution obtained in (iii) from any precipitate formed to obtain a separated solution; and
(v) diluting the separated solution obtained in (iv) with a solvent comprising water to obtain the anti-adsorption solution.

20. The process of claim 19, wherein:
the solution prepared in (i) further comprises from 0.1% to 10% m/V of a first acid;
the organic solvent solution in (ii) comprises 0.1% to 10% m/V of a second acid; and
the solvent of (v) further comprises from 0.1% to 10% m/V of a third acid.

21. The process of claim 19, further comprising heating the diluted solution obtained in (ii) prior to cooling the diluted solution in (iii).

22. The process of claim 21, wherein the diluted solution is heated for 1 minute to 1000 minutes to a temperature of 30° C. to 120° C.

23. The process of claim 21, wherein the diluted solution is heated for 1 minute to 10 minutes to a temperature of 75° C. to 100° C.

24. The process of claim 19, wherein the concentration of the one or more proteins and/or peptides in the solution in (i) is 25 μg/mL to 25 mg/mL, and the solution in (i) further comprises 0.1% to 10% m/V of a first acid.

25. The process of claim 19, wherein the solution prepared in (i) further comprises greater than 0% to 1% m/V of a first acid.

26. The process of claim 19, wherein in the diluted solution is cooled for greater than 0 minutes to 600 minutes to a temperature of 40° C. to −35° C.

27. The process of claim 19, wherein the diluted solution is cooled for 5 minutes to 60 minutes to a temperature of 8° C. to −8° C.

28. The process of claim 19, wherein the solvent of (v) further comprises 0.1% to 1% (m/V) of a third acid.

29. The process of claim 28, wherein in (v) 0.1 to 9.9 parts of the separated solution is diluted with 9.9 to 0.1 parts of the solvent.

30. The process of claim 19, wherein the one or more proteins or peptides are selected from the group consisting of albumin, ovalbumin, lactalbumin, globulin, gelatin, and a combination thereof.

31. The process of claim 29, wherein the one or more proteins or peptides is albumin.

32. The process of claim 20, wherein the first acid, the second acid, and the third acid are each independently selected from formic acid, acetic acid, or trifluoroacetic acid.

33. The process of claim 20, wherein the first acid, the second acid, and the third acid are formic acid.

34. The process of claim 19, wherein the organic solvent solution comprises acetonitrile, methanol, isopropanol, or denatured ethanol.

35. The process of claim 19, wherein the organic solvent solution comprises acetonitrile.

36. A method for analyzing a peptide, the method comprising:
adding a sample to the anti-adsorption solution prepared by the process according to claim 19; and
analyzing the sample for the presence of peptides.

37. The method of claim 36, wherein the sample is a serum sample.

38. The method of claim 36, wherein analyzing the sample for the presence of peptides comprises performing an analytical method selected from the group consisting of immune-based protein analysis methods, liquid phase chromatography, mass spectrometry, and peptidomics.
